(19) United States
(12) Patent Application Publication
(10) Pub. No.: US 2026/0286313 A1
(43) Pub. Date: Sep. 24, 2026

(54) CREATING BIOLOGICAL FIBERS FOR TISSUE CONSTRUCTS

(71) Applicant: Regents of the University of Minnesota, Minneapolis, MN (US)

(72) Inventors: Caleb Darwin Vogt, Brooklyn Park, MN (US); Angela Panoskaltsis-Mortari, Woodbury, MN (US); Anthony Vitale, Iowa City, IA (US); Alexander Paulsen, Minneapolis, MN (US); Siddhant S Yelwande, Minneapolis, MN (US); Joseph Robert Broomhead, Southport, NC (US); Johanna Teegarden, Lakeland, MN (US); Kyle Kunisaki, Eagan, MN (US)

(21) Appl. No.: 19/474,597

(22) PCT Filed: Apr. 11, 2024
(86) PCT No.: PCT/US2024/024122

§ 371 Date: Oct. 10, 2025

Related U.S. Application Data
(60) Provisional application No. 63/495,428, filed on Apr. 11, 2023.
(60) Provisional application No. 63/498,462, filed on Apr. 26, 2023.

(51) Int. Cl. C12N 5/071; B33Y 10/00; B33Y 70/00; C12N 5/00

(52) U.S. Cl. CPC C12N 5/0691; B33Y 10/00; B33Y 70/00; C12N 5/0068; C12N 2502/1347; C12N 2513/00

(57) ABSTRACT

Devices, systems, and techniques are described for creating pre-aligned cells in fibers to be wound into tissue constructs or microtissues that can be printed into larger tissue constructs. For example, a method includes dispensing a biological fiber into a solution, wherein the biological fiber comprises a plurality of cells within a hydrogel. The method may then include spooling, under tension, the biological fiber from the solution and onto a spool structure. In some examples, the method may also include winding the biological fiber around a tissue frame to create a tissue construct or cutting the biological fiber matured into a pre-aligned microtissue from the spool structure and used in a bioink for printing tissue constructs.

This PCT application claims priority to, and the benefit of, U.S. Provisional Patent Application No. 63/495,428, filed on Apr. 11, 2023, and U.S. Provisional Patent Application No. 63/498,462, filed on Apr. 26, 2023, the entire contents of these applications are incorporated herein by reference.

### TECHNICAL FIELD

This disclosure relates generally to methods, systems, and devices for engineering tissue.

### BACKGROUND

Biological tissues are highly ordered by means of cell layers, cellular alignment, or both. The precise structural arrangement of cells is critical to all tissue function.

Mechanically active tissues including skeletal muscle, smooth muscle, cardiac muscle, tendons, and ligaments are all soft tissues structured with elongated cells that are aligned in the direction of the greatest stress. In long skeletal muscles and tendons, all cells are aligned in a single direction. However, cardiac muscle and the smooth muscle of the gastrointestinal and urogenital systems have layers of muscle with multiple directions of alignment.

### SUMMARY

This disclosure describes example techniques and systems for making and using biological fibers or microtissues that include pre-aligned cells for creation of larger tissue constructs. The biological fibers may be later wound to form a tissue construct or microtissues (e.g., biological fibers cut into smaller segments) can be included in a bioink for three dimensional (3D)-printed model biological microenvironments. The biological fibers (and microtissues) may be pre-aligned in that the biological fibers are formed using cells that align in one direction. The cells may be initially disposed in a core surrounded by a shell that may include a gel. The structure of cells within the shell may be expelled into a solution to create the biological fiber that is disposed onto a spool structure for storing and/or maturing. In some examples, the biological fiber may be subjected to tension before and/or during when the biological fiber is wrapped around the spool structure. As some examples, the system may then apply a tension to the biological fiber, such as via a spool or intermediate roller, and spool the biological fiber onto the spool under the tension. This tension may enable the cells to “pre-align” in the direction of the tension on the spool. The biological fiber may be referred to as a pre-aligned microtissue since the cells may align themselves in the direction of the tension. From the spool, the biological fiber may be used for different purposes.

The biological fibers may be used in long lengths or cut into smaller “microtissues” that still include the pre-aligned cells (which may be described as pre-aligned microtissues). In some examples, the method may include suspending the pre-aligned microtissues in a liquid (e.g., hydrogel) material to create a “bioink,” and printing the bioink with microtissues using a 3D extrusion bioprinter. Laminar flow physics and geometric constraints cause the microtissues to align with each other in the same or different direction as they are extruded out of the nozzle of the printer; resulting in direct writing of aligned cellular bundles. This enables for creation of complex layers and patterns having any desired alignment of cells. In this manner, the pre-aligned microtissues may be assembled into larger, macroscale tissues using technologies including 3D-bioprinting. A scaffolding material (e.g., a biocompatible polymer or biopolymer) may be used to create a desired shape, and the microtissues with pre-aligned cells can be added to create an immature “tissue construct.” The tissue construct may then be matured in a bioreactor that applies chemical, electrical, and mechanical stimulation to cause maturation of the tissue into the desired final form, such as connective tissue that includes smooth or skeletal muscle, tendons, ligaments, or any other tissues. Indeed, this process may be configured for creation of all types of cellular alignment found in muscle, tendon, and ligament tissues in the body, including the highly complex structures found in the cardiovascular, gastrointestinal, and urogenital systems.

In some examples of the present disclosure, the method may include winding biological fibers that may have been previously spooled to form a tissue construct, where the biological fibers may include cells and, in some examples, pre-aligned cells before the winding. The biological fibers may be acellular to serve as spacers, depending on final design requirements, or may include growth and differentiation factors in some examples. This winding process may use and generate pre-aligned biological fibers that are wound into larger, macroscale tissue constructs such as muscles, organs, or other tissues. In some examples, the cells within the biological fiber may align in the direction of the tension on the spool structure prior to winding. In some examples, tension applied to the biological fiber after the fiber is wound on a tissue frame may also, or alternately, cause the cells of the biological fiber to align in the direction of the tension. The tissue construct may then be matured in a bioreactor that applies chemical, electrical, and mechanical stimulation to cause maturation of the tissue into the desired final form, such as connective tissue that includes smooth or skeletal muscle, tendons, ligaments, or any other tissues.

The biological fiber may be pre-aligned in that the fibers and/or tissue constructs are formed using cells that align in one direction. The cells may be initially disposed in a core surrounded by a shell that may include a gel. The structure of cells within the shell may be expelled into a solution to create a biological fiber that is disposed onto a spool structure for storing and/or maturing.

In some examples of winding the biological fiber to form a tissue construct, the system may include a robotic arm with several degrees of freedom, such as six or more degrees of freedom. With a tissue frame attached to the end of the robotic arm, the robotic arm may move the tissue construct in a predetermined spatial path that pulls the biological fiber from a spool and lays down the biological fiber on the tissue frame in a target pattern. This target pattern may provide for the desired directionality of the biological fiber for a functional tissue construct. In other examples, the robotic arm may move the biological fiber with respect to a tissue frame (that is stationary or also moving) to wind the biological fiber around the tissue frame. This enables the creation of complex layers and patterns having any desired alignment of cells. This process also enables the creation of all types of cellular alignment found in muscle, tendon, and ligament tissues in the body, including the highly complex structures found in the cardiovascular, gastrointestinal, and urogenital systems.

In some examples, a method includes dispensing a biological fiber into a solution, wherein the biological fiber comprises a plurality of cells within a hydrogel, and spooling, under tension, the biological fiber from the solution and onto a spool structure.

In some examples, a method includes dispensing a biological fiber into a solution, wherein the biological fiber comprises a plurality of cells within a hydrogel; applying tension to the biological fiber; and spooling, under the tension, the biological fiber from the solution and onto a spool structure.

In some examples, a system includes a spinneret configured to dispense a biological fiber into a solution, wherein the biological fiber comprises a plurality of cells within a hydrogel; a spool structure configured to store the biological structure; and a motor coupled to at least one of a roller or the spool structure and configured to apply tension to the biological fiber, wherein the motor is configured to assist in spooling, under the tension, the biological fiber from the solution and onto the spool structure.

In some examples, a method includes removing a biological fiber from a spool structure, wherein the biological fiber comprises a plurality of cells within a hydrogel; and winding, with a robotic arm, the biological fiber around the tissue frame.

In some examples, a system includes memory configured to store a predetermined spatial path and control circuitry configured to control a spool structure to remove a biological fiber from the spool structure, wherein the biological fiber comprises a plurality of cells within a hydrogel; and control a robotic arm to wind the biological fiber around a tissue frame.

The details of one or more embodiments of the disclosure are set forth in the accompanying drawings and the description below. Other features, objects, and advantages of the disclosure will be apparent from the description and drawings, and from the claims.

### BRIEF DESCRIPTION OF DRAWINGS

FIG. 1 is a graphical representation of an example gastroesophageal junction (GEJ) GEJ fascicle arrangement with a thick inner circular muscle layer of the esophagus continuous with bundles of smooth muscle in accordance with the examples of this disclosure.

FIG. 2 is a graphical representation of pre-aligned microtissues in accordance with the example of this disclosure.

FIG. 3A is a flow diagram of an example technique for generating a biological fiber that can include or promote aligned cells.

FIG. 3B is a flow diagram of an example technique for creating pre-aligned microtissues and larger tissue constructs.

FIGS. 4A, 4B, and 4C are example cross-section and profile views of example fibers generated using a core-shell spinning technique.

FIGS. 5A and 5B are conceptual diagrams of an example spinning device for generating microtissue structures.

FIG. 6A is a conceptual diagram of an example system for spooling biological fibers in a crosslinking bath.

FIG. 6B is a conceptual diagram of an example system for spooling biological fibers in a collection bath separate from a crosslinking bath.

FIG. 7A is a conceptual diagram of an example expandable device for creating tension on biological fibers.

FIGS. 7B, 7C, 7D, and 7E are different views of the expanding device of FIG. 7A.

FIGS. 7F and 7G are different views of a gear of expanding device of FIG. 7A.

FIGS. 7H, 71, 7J, and 7K are different views of an expandable device for creating tension on biological fibers.

FIG. 8 is a flow diagram of one example of cutting biological fibers from a loaded spool.

FIGS. 9A and 9B are conceptual diagrams of an example system for cutting biological fibers from a loaded spool.

FIG. 10 is a flow diagram of an example technique for spooling biological fiber and then cutting the spooled biological fiber to specific lengths.

FIG. 11 is a conceptual diagram of an example system for winding biological fibers around a tissue frame to create a tissue construct.

FIG. 12 is a conceptual diagram of distance restraints for an example robotic arm.

FIGS. 13A and 13B are conceptual diagrams of example connections from a robotic arm to a tissue frame.

FIG. 14 is photograph of an example tissue frame for creating a tissue construct of a GEJ.

FIGS. 15A and 15B are front and side views of an example spool structure and bath from which a robotic arm winds the biological fiber.

FIG. 16 is a front view of the bath and example robotic arm coupled to a tissue frame.

FIG. 17 is an image of an example tissue frame with string wound around the tissue frame to illustrate possible directions for a biological fiber on the tissue frame.

FIG. 18 is a flow diagram of an example technique for winding biological fiber onto a tissue frame to create a tissue construct.

FIG. 19 is a flow diagram of an example technique for creating tissue constructs using spooled biological fiber.

### DETAILED DESCRIPTION

In general, this disclosure describes examples of tissues and fibers that are created with cells aligned prior to creating a tissue construct. These biological fibers can be used to create 3D tissue constructs or cut into smaller microtissues and used in a bioink to construct 3D-printed biological environments.

Biofabrication and tissue engineering have particularly made strides in replacing and improving tissues for living organisms. Extrusion methods, inkjet and droplet printing, and laser assisted printing, have allowed for high accuracy and repeatability in printing matrices of organs and tissues specific to living beings. However, integrating these printed tissue matrices poses challenges, including the complexities of existing body structures, tissue structure, tissue orientation, attachment methods, and maintaining cell viability. Several methods have been developed to address these challenges, but each comes with its own difficulties.

In addition, biological tissues are highly ordered by means of cell layers, cellular alignment, or both. As one example, natural muscle tissue generally exhibits a high degree of alignment that allows for efficient directional force generation. The precise structural arrangement of cells is important to all tissue function. Mechanically active tissues including skeletal muscle, smooth muscle, cardiac muscle, tendons, and ligaments are all soft tissues structured with elongated cells that are aligned in the direction of the greatest stress. In long skeletal muscles and tendons, all cells are aligned in a single direction. Cardiac muscle, the smooth muscle of the gastrointestinal system, and the urogenital systems have layers of muscle with multiple directions of alignment. Recreating the more complex structures found in these cardiac and smooth muscle tissues may be very difficult. Indeed, cell alignment is a challenge in tissue engineering.

Cellular alignment, in one direction, may be performed using geometric cues, remote fields, passive mechanics, and active mechanics. For example, as descried herein, cells may be pre-aligned by “wet-spinning” and applying tension to the cells. The cells may be initially disposed in a core surrounded by a shell that may include a gel. The structure of cells within the shell may be expelled into a solution to create a biological fiber suspended within the solution. The structure that creates and expels this biological fiber may be referred to as a spinneret. The system may then apply a tension to the biological fiber, such as via a spool or intermediate roller, and spool the biological fiber onto the spool under the tension. This tension on the spool may enable the cells to “pre-align” in the direction of the tension on the spool. In some examples, the tension may be created by utilizing gravity and/or a motor that rotates a roller or the spool structure. The system may measure the tension in some examples and use that tension to control the motor in a closed-loop control system. This process creates very long and thin fibers that the cells grow along, which causes them to align with each other on the spool structure. Once the fiber is formed, it is cut into small pieces and put into a bioprinter. The biological fiber that includes the cells is about the thickness of a single hair (50 micrometers), so very long fibers are needed to get enough tissue volume for printing. These biological fibers can be longer than a football field (over 100 meters), so spooling the fiber around the spool structure during the wet-spinning process can help to avoid tangling of the biological fiber.

In some examples, the system can wind the biological fiber onto a specialized spool structure during the spinning process. The described spooling process may have certain features or advantages. First, in some examples, this wet-spinning can take place in a fluid bath (e.g., within one or more solution baths). The biological fiber can be prone to drying, which would kill the cells inside. Spinning such a long fiber can take 20 minutes or more, at which point the water contained in the fine biological fiber will have evaporated significantly. In conventional wet-spinning, a dry fiber is desirable. However, it would defeat the purpose of making a living cell fiber in a dry environment. As described herein, the spooling system configured to spool the fiber while it is still in the wet-spinning bath can enable the cells to survive the spooling process to form the pre-aligned microtissues. In some examples, the spool structure may be only partially in the solution, or completely out of the solution, but the system may apply the solution or other fluid to biological fiber to maintain hydration in the biological fiber. Second, cells in the fiber can be affected by the tension that is being applied during the winding process. Too little tension may cause the cells not to line up very well. Too much tension could lead to the fiber breaking frequently, which would lead to an inefficient production process. The system may thus monitor the tension used to spool the biological fiber. Finally, the system must work in a sterile environment to avoid contamination with bacteria and mold. The devices and machines used in the production can be sterilized by very high heat and pressure, gas, or chemical treatments, but previous winding systems for non-biological materials are not designed to be sterilized to enable such sterilization.

Wet-spinning of cell fibers as described herein is an emerging tissue production method with the potential to solve many problems the field faces. Efficient production and handling of these biological fibers will enable for the creation of new products and testing platforms in industry and academia. This described spooling or winding system can provide the means to scale up biological fiber production for bioprinting tissue constructs such as organs or other tissue portions. For example, the biological fiber may be wound around a tissue frame to create a tissue construct or the biological fiber may be cut into smaller fibers (e.g., pre-aligned microtissues) that can be used in a bioink for 3D printing tissue constructs with the microtissues.

In some examples of the present disclosure, a system may create pre-aligned microtissues and then incorporate these pre-aligned microtissues into a 3D bioprinting “bioink” to recreate complex muscle structures. Examples of the present disclosure disclose methods and systems to create pre-alignment of cells and resulting microtissues that may then be assembled into larger, macroscale tissues using technologies including 3D-bioprinting. Microtissues that contain cells that are all aligned within the microtissue structure may be subassemblies that are then assembled into larger tissues. These microtissues may be used to form the desired alignment structures including complicated tissue structures such as the GEJ.

The printing process may be performed by creating a desired shape out of a scaffolding material (e.g., typically made from a biocompatible polymer or biopolymer), and to that cells of the pre-aligned microtissues are then added. This creates an immature “tissue construct.” This construct may then be matured in a bioreactor that applies signals including chemical, electrical, and mechanical stimulation to cause maturation of the tissue into a desired final form.

In examples of the present disclosure, bioreactor signals, including chemical, electrical, and mechanical stimulation, may be applied at the beginning of the process rather than the end. Mature microtissue “building blocks” using cells are created up front. A scaffold may then be deposited with microtissues in the desired shape. The microtissues are then matured in a bioreactor. This unidirectional cell alignment may be applied on the micron scale to create small pieces of pre-aligned tissue. These small tissues can then be assembled into larger aligned tissues through the use of three-dimensional (3D) bioprinting technology. In one example, active mechanics may be used to create small, cylindrically shaped pre-aligned microtissues with approximate dimensions of 2 mm in length and 100 microns in width. In other examples, the pre-aligned microtissues may be smaller than 2 mm in length, such as around 300 micron. Regardless of the length that is used, these microtissues, that are longer than they are wide, may be made of cells aligned with the primary axis of the microtissue.

These microtissues may then be suspended in a hydrogel material to create a bioink that may be used in a 3D bioprinter. Laminar flow physics and geometric constraints can cause the microtissues to align in the direction of flow and/or with each other as they are extruded out of the nozzle of the printer, resulting in direct writing of aligned cellular bundles. Examples of the present disclosure enable for creation of complex muscle layers and patterns of alignment. This enables for recreation of all types of cellular alignment found in muscle, tendon, and ligament tissues in the body, including the highly complex structures found in the cardiovascular, gastrointestinal, and urogenital systems.

In some examples described herein, cell fiber winding (e.g., winding of one or more biological fibers to form a tissue construct) is a method that addresses various issues in the integration of printed tissues for some structures. Long strands of cell fibers (e.g., fibers prepared with pre-aligned cells on a spool) are wound onto tissue frames that are integrated into the body and dissolved, in some examples, leaving behind tightly wound fibers that act as the tissue itself. The tissue frames may also be dissolved, or otherwise removed, immediately after the winding process or after an intermediate maturation process in an incubator or bioreactor. However, winding these delicate cell fibers within the biological fiber can require a controlled and monitored method, with consideration for alignment and moisture levels. Maintaining tension during winding is also an important aspect, making the process complicated. Automation and controlled parameterization can provide robust systems or processes for the biological fiber winding process and eventual tissue construct generation. In the case of cell fiber alignment and winding, such automation can be achieved through the development of a device that can move in various orientations to wind and align the cell fibers into any desired pattern and structure.

In some examples, a robotic arm (e.g., a manipulator) can be a main component for the design of the system. A robotic manipulator arm with 6 degrees of freedom (DoF) can be used to hold the tissue frame for an example organ (e.g., a tissue construct) in place and can rotate the frame in multiple axial orientations to wind the biological fiber around the frame.

For fiber management, the robotic arm may create a gravity-based tension management system that can create minimum electronic and mechanical isolation between the collector spool for cell fiber and the robotic winding arm. This fiber management can also take into consideration the addition of force sensors to measure the amount of stress applied on the fiber while it undergoes multiple axial rotations. The system can use the force sensors as feedback for controlling the movement, such as speed, of the robotic arm as it moves the tissue frame. This can keep the speed in desired range, which can be another important factor in the entire winding process.

For moisture management, the system may be configured to maintain conditions to keep the biological fibers sufficiently moist (e.g., hydrated) during some or all of the winding process. The liquid selected for this purpose could be is calcium chloride if alginate is used in the hydrogel, but other liquids or solutions may be used in other examples. This liquid can also act as an adhesive to stick these biological fibers onto the surface of the tissue frame. The moisture management process can include a mixture of two subprocesses that involve drip moisturizing of the fiber and liquid baths to keep the fiber hydrated and maintain cell health and viability.

An arm microcontroller can be used to move the robot arm according to a desired path in space and also used to program the robot for required orientations. The controls of this device can also include a user interface (UI) which can provide a choice to a user to select the type of tissue or scaffold to be wound with the biological fiber. The controls can also include servo angle measurement to replicate this device on any scale required.

The systems and techniques described herein can provide a multi-axial orientation device that can act as a practical actuating media for the process of cell fiber winding. The device can be configured to wind long strings of muscle fibers onto organ frames with respect to multiple axes automatically, taking into account aspects such as modeling, control, manipulation, and peripherals for fully automated multi-axial cell fiber winding.

The multi-axial winding device and system described herein can be utilized for biological fibers, such as smooth muscle cell fibers, and enables the process of bio fabrication using the working principles of robot arm, tension and peripherals. The multi-axial winding device can lay down biological fibers in different directions such that cells of the biological fibers can be orientated in any desired direction. In this manner, tissue constructs can be generated by this system to recreate various organs or tissues, or develop repair tissues with desired characteristics.

The tissue construct created by the winding of the biological fiber onto the tissue frame can then be matured in a bioreactor that applies signals including chemical, electrical, and mechanical stimulation to cause maturation of the tissue into a desired final form. The biological fibers may be cylindrically shaped and pre-aligned microtissues with approximate dimensions of 100 microns in width, but smaller or larger widths may be used in other examples. Examples of the present disclosure enable for creation of complex muscle layers and patterns of alignment. This enables for recreation of all types of cellular alignment found in muscle, tendon, and ligament tissues in the body, including the highly complex structures found in the cardiovascular, gastrointestinal, and urogenital systems.

Cancer of the gastrointestinal system, such as gastroesophageal adenocarcinoma, is a significant source of morbidity and mortality, with an estimated 200,000 new cases in the United States in 2019. For cancers involving the alimentary canal (e.g., esophagus, stomach, intestines), surgical resection of the cancer and anastomosis of the cancer-free portions of the tube is the best treatment if the tumor is small. The GEJ may be treated in this manner with a surgery known as gastric pull-up; however, patients may experience complications after the procedure. Loss of the GEJ's anatomical barrier to reflux may lead to pulmonary complications. Thus, while simple resection and anastomosis is attractive in some regions of the alimentary canal, there is an urgent need for new solutions at the GEJ. Recent developments in induced pluripotent stem cells raise the possibility that patient-specific autografts may be tissue-engineered. Clinical trials have successfully replaced portions of the esophagus with tissue-engineered products. Despite these apparent successes, almost no work has been done to rebuild the GEJ. Of the hurdles preventing the field from progressing, anatomical complexity of the muscles structure and the need for vascularization are the most pressing. As described herein, pre-aligned microtissues used for 3D printing of larger tissue constructs may enable the creation of various anatomical structures, such as the GEJ. 3D printing of pre-aligned microtissues may enable the creation of any other types of tissues in other examples. Alternatively, the biological fibers may be wound around a tissue frame representing the GEJ (or other organ or structure) to form a tissue construct with pre-aligned cells of the biological fibers in order to create a representation of the GEJ.

Generally, the microtissues described herein may be comprised of mammalian cells (human or non-human). However, in other examples, the techniques described herein may also be performed using non-mammalian cells, such as plant or insect cells. In some examples, plant cells may be used to generate various constructs suitable for pharmaceuticals and/or delivery of pharmaceuticals.

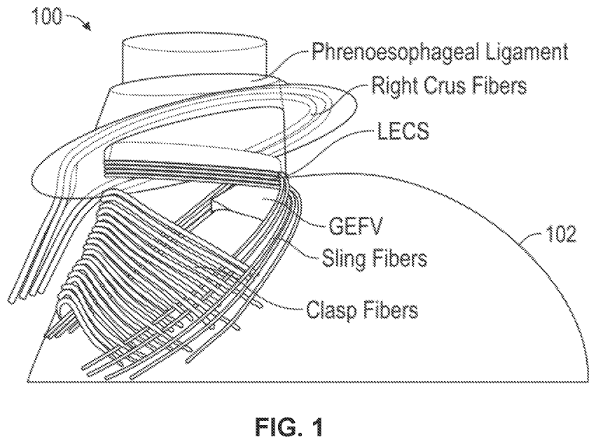

FIG. 1 is a graphical representation of an example gastroesophageal junction (GEJ) 100 fascicle arrangement in accordance with the examples of this disclosure. The GEJ 100 differs from the rest of the alimentary canal in its complex arrangement of smooth muscle bundles. At the GEJ 100, there is a thickening of the inner circular muscle layer of the esophagus that is continuous with bundles of smooth muscle in the stomach wall known as gastric sling fibers. These fibers are opposed by gastric clasp fibers on the lesser curvature of stomach 102. All three of these components have specific mechanical roles and contract in unison to maintain the anti-reflux barrier at the GEJ 100. A feature to achieving contractile function is highly aligned gut smooth muscle cells (gSMCs) within these bundles. As described further below, printing pre-aligned microtissues into larger tissue constructs may enable creating of the GEJ 100 with highly aligned gSMCs that provide the functionality of the GEJ 100.

Over 200,000 people in the United States are predicted to develop cancer of the alimentary tract (e.g., the esophagus, stomach, intestines) in the United States in 2019. Surgical resection is often the preferred method of treatment, but it may lead to loss of normal function within the tract. A key anatomical region is the GEJ, that performs one-way valve function to keep stomach contents from refluxing into the esophagus. Surgical removal of the GEJ and anastomosis of the stomach to the esophagus in a gastric pull-up procedure is associated with significant morbidity and mortality, with up to 20% of patients experiencing respiratory complications post-surgery due to stomach contents rising high into the upper esophagus and into the trachea as micro-aspiration. Tissue-engineered solutions may be ideal for replacing the GEJ, but as discussed there are numerous challenges. Recreating the complex arrangement of gSMC bundles at the GEJ may not be done with current methods, and vascularization of tissue-engineered constructs remains a challenge. The individual gSMCs at the GEJ exhibit a high level of alignment within the bundles, allowing each bundle to maximize its force production. These bundles are wrapped in numerous configurations around the GEJ and work together to maintain a high-pressure zone that resists reflux. Because this tissue is metabolically active, it requires substantial blood flow. 3D bioprinting can make medium vascular channels (e.g., 200 μm diameter) but efficiently making capillaries (e.g., 8 μm diameter) to increase the surface area required for mass transfer remains difficult. As described herein, printing microtissues with pre-aligned cells supplemented with vascular cells may enable the creation of such smaller blood vessels and capillaries that may be able to provide blood flow to the larger printed tissue construct.

Examples of this description include an approach that re-arranges the traditional tissue engineering process. Some methods to align muscle cells or create capillary vasculature can work on small tissue sizes but may not be scaled-up to produce large constructs. This is because the traditional approach is to add cells into a scaffold that provides shape, then mature the construct in a complex bioreactor. This may not work for the GEJ or other structures, because of the complex alignment of gSMC bundles. Furthermore, creating capillaries traditionally relies on placing endothelial cells in the constructs and hoping they may form a network before necrosis sets in at the center of thick tissues. Rather than scaling-up the size of existing methods, examples of the present disclosure produce pre-aligned and pre-vascularized microtissues, that may then be used in a 3D bioprinter as “building blocks” to make larger tissue constructs or automated winding process to make larger tissue constructs. This approach may move much of the tissue maturation process to the beginning of the process, such as in the creation of the initial biological fibers that can be matured into pre-aligned microstructures with pre-aligned cells that can be used in bioink, rather than the end. This pre-alignment of cells may greatly decrease the complexity during final maturation in a bioreactor. Bioprinting of pre-matured microtissues (and in some cases including pre-aligned cells) may result in enhanced control over tissue alignment and vascularization.

In some examples, bioprinting pre-aligned microtissues may result in greater alignment and active force generation in the aligned direction when compared to traditional bioprinting. The microtissues may be formed by strain-induced (e.g., mechanical cues to the cells) alignment of gSMC-laden collagen hydrogels. The phenotype of the gSMCs in the microtissues and overall tissue maturity may be assessed by expression of key proteins. Similarly, winding biological fibers with pre-aligned cells can improve alignment of cells in the direction of tension and also create tissue constructs with cell alignment to achieve function more similar to biologically original structures or organs.

Printing a tissue with pre-vascularized microtissue bio-ink may result in faster development of capillary networks compared to traditional freely suspended cell bioprinting. Microtissues with gSMCs and fluorescent human umbilical vein endothelial cells (hUVECs) may be made by spheroid culture. The effects of gSMCs and culture conditions on sprouting of hUVECs from the spheroid into a collagen gel may be measured. These microtissues may be printed between two perfusable large vascular channels and cultured to form a capillary network that connects the two larger channels. Dextran diffusion through the network may be used to compare flow to control, that may be a traditional 3D bioprinting of gSMCs and endothelial cells suspended as individual cells within the bioink.

Traditional tissue engineering relies on first producing a scaffold that defines a shape that may be populated by cells. This combination of cells and scaffold is then placed into a custom bioreactor to try to provide the necessary cues to form the final matured structure. Traditional extrusion bioprinters use a “bio-ink” made from a hydrogel precursor loaded with individually suspended cells that is extruded out of a nozzle to create the cell-laden structure.

Examples of the present disclosure disclose reimagining the conventional workflow of tissue engineering. These aims may be combined to provide the field with a new, unified approach to generating smooth muscle. These examples may be broadly applicable to tissue engineering in the alimentary tract and may apply to other challenges such as establishing neural control. Examples of the present disclosure may be extended to applications in other aligned, vascularized tissues, such as cardiac muscle.

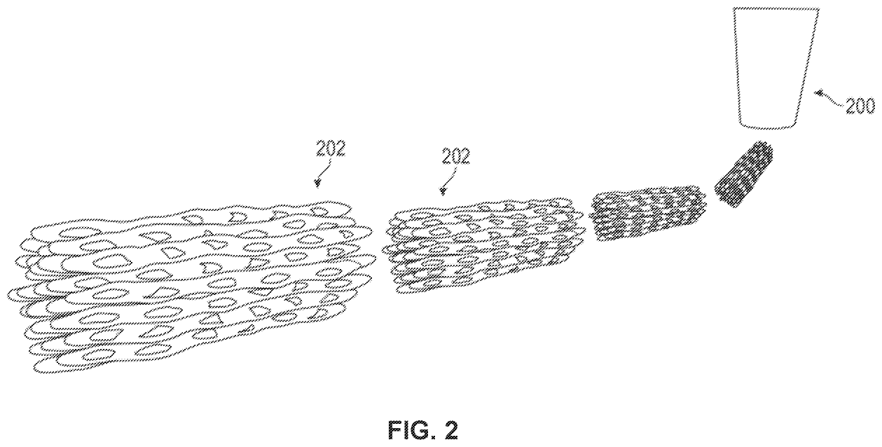

FIG. 2 is a graphical representation of pre-aligned microtissues 202 in accordance with the example of this disclosure. The problem for creating the GEJ using such a workflow is that making a bioreactor to achieve the complex anatomical structure and also induce vascularization is prohibitively complex. Examples of the present disclosure may rearrange existing techniques to create mature microtissues, accomplishing the most complex parts of the process while the tissues are still on a manageably small scale. These pre-matured and pre-aligned microtissues 202 (e.g., such as microtissues created by cutting biological fiber described herein) may then be used as the building blocks to create larger tissues that do not require a complicated bioreactor to achieve alignment and vascularization. For the purpose of printing aligned gSMC bundles, an advanced bio-ink may be made from pre-aligned microtissues 202, rather than individually suspended cells as is done in traditional bioprinting. A combination of geometric constraints and fluid flow alignment may be used to print the microtissues 202 end-to-end as they are released from printing nozzle 200. In some examples, microtissues 202 can then fuse to form a fascicle or bundle of structures. Examples of the present disclosure disclose a tissue engineering method allowing for continuous extrusion bioprinting of smooth muscle bundles that are similar to those found in the native GEJ. As described further below, the pre-aligned microtissues may be created via applying a tension to biological fibers that include the cells which align themselves under the tension.

To address the vascularization problem, pre-vascularized gSMC microtissues may be created by co-culturing gSMCs with endothelial cells in pre-aligned microtissues. Such a combination of pre-vascularized and pre-aligned microtissues have not been applied before in bioprinting. These pre-vascularized microtissues may be suspended in a bio-ink and used to print.

Bioprinting pre-aligned microtissues may result in greater alignment and active force generation in the aligned direction when compared to traditional bioprinting. Cell alignment is important for muscular contraction on the tissue level, because muscle tissue is largely incompressible and therefore may not change shape if contracted in all directions at once. A simple method to achieve cell alignment in tissue is through mechanical strain induction. Smooth muscle cells may be highly sensitive to strain and tend to align either parallel or perpendicular to applied strain, depending on various factors. 3D tissues with aligned cells may be formed by suspending cell-laden hydrogels in small wells between rubber posts. As cells remodel the hydrogel, they cause the gel to undergo compaction, inducing a strain in the gel between the two anchoring posts. The cells align with their long axis parallel to the strain, resulting in highly aligned tissues. When released from the anchoring posts, this may yield cylindrically shaped microtissues of aligned cells that may be suspended in a bio-ink for printing.

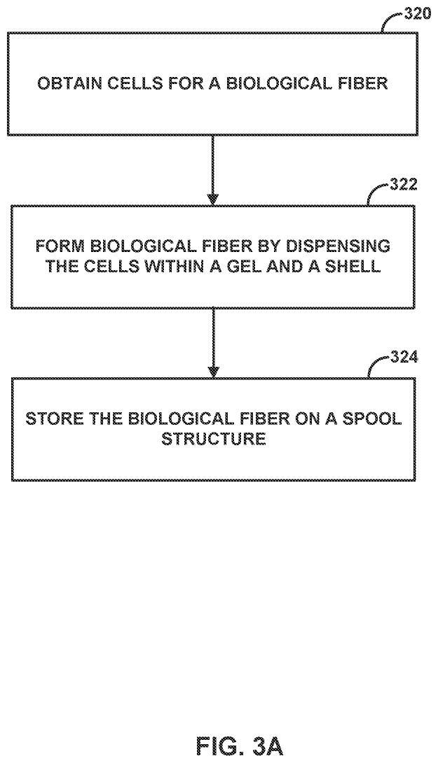

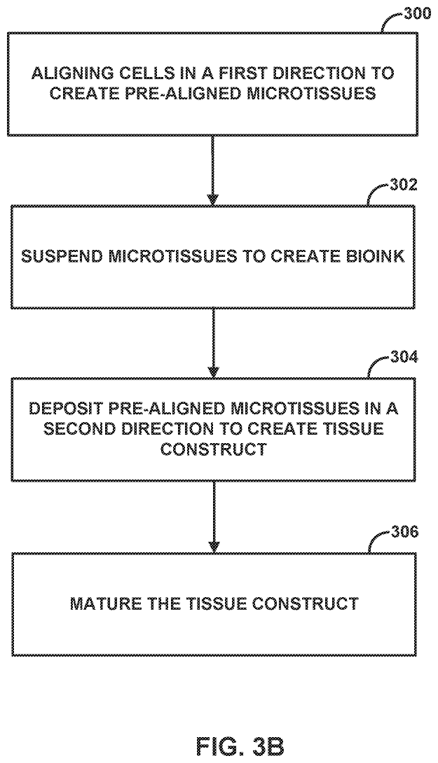

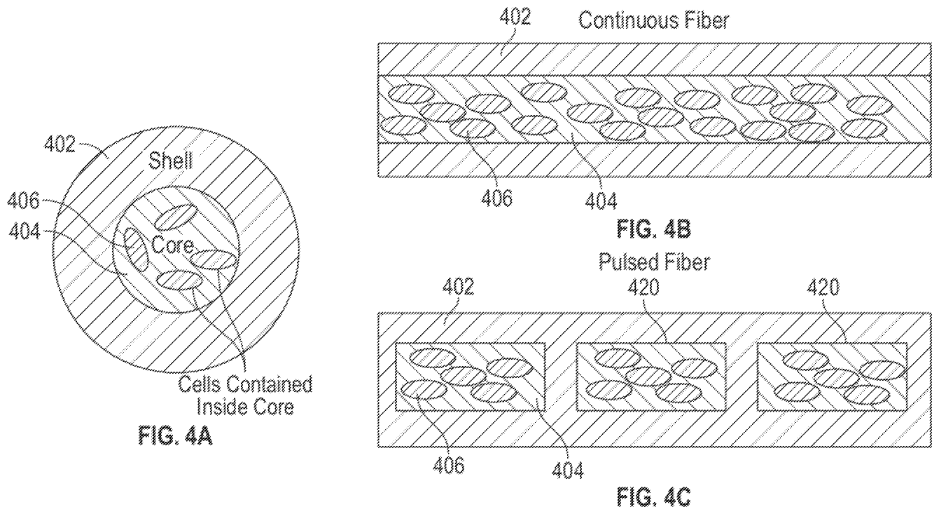

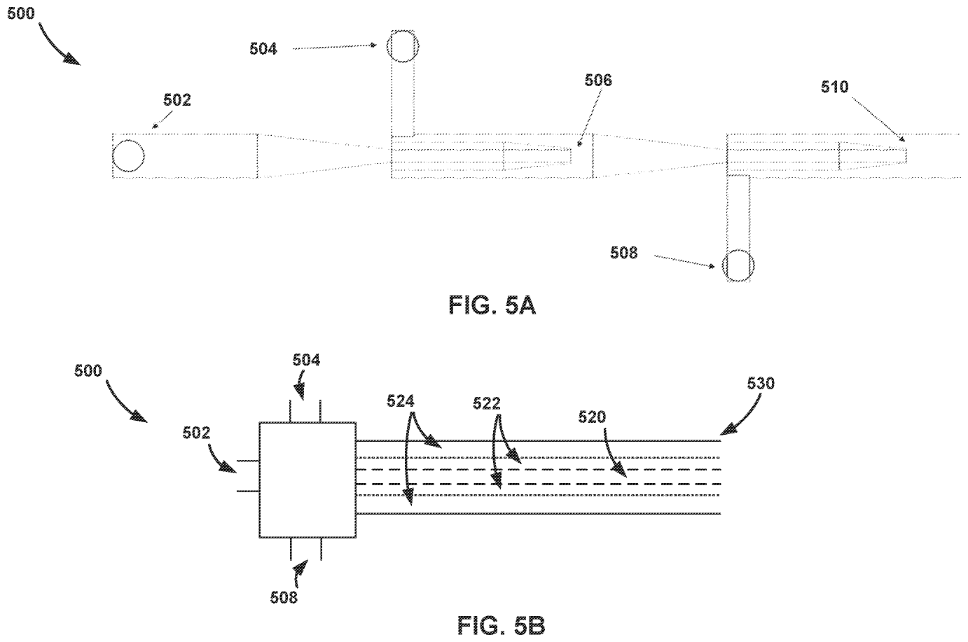

FIG. 3A is a flow diagram of an example technique for generating a biological fiber that can include or promote aligned cells in accordance with the present description. The biological fiber may then be cut into smaller pieces to create pre-aligned microtissues or the biological fiber can be wound into desired tissue constructs. In the example of FIG. 3A, a system, such as spinning device 500 in FIG. 5, can obtain cells for a biological fiber. These cells may be specific types of cells for a resulting tissue construct, such as muscle cells for a GEJ (320). Spinning device 500, for example, can then form a biological fiber by dispensing the cells within a gel and surrounded by a shell (322). The resulting biological fiber may be similar to the biological fiber described in FIGS. 4A-4C. In some examples, these fibers may be created to have widths or diameters from approximately 100 μm to approximately 200 μm, but smaller or larger widths may be used in other examples. After the biological fiber is formed by spinning device 500 or some other device, the biological fiber is stored on a spool structure or other device that can store the biological fiber before the winding process or being cut into microtissues (324). In some examples, the biological fiber may be subject to tension when stored on the spool structure to reduce fiber entanglement and/or subject the cells within the fiber to strain which may cause the cells to further align in the direction of the tension.

In one example, muscle cells of the fiber may be matured using bioreactor signals so that the muscle cells are aligned in the first direction to form biological fibers or microtissues with pre-aligned cells. For instance, a system may produce cells within a gel in a biological fiber. This biological fiber may initially include muscle cells which are not yet aligned. The biological fiber may then be spooled, under tension, to a spool structure. While the biological fiber is on the spool structure under tension, the cells remodel the hydrogel and may cause the gel to undergo compaction, inducing a strain in the hydrogel along the direction of the tension on the spool structure. The muscle cells can then align with their long axis parallel to the strain (or tension), resulting in highly aligned microtissues. When released from the spool structure, the resulting biological fiber includes pre-aligned cells and may be ready to be wound around a tissue frame or cut into pre-aligned microtissues as described herein.

The larger tissue construct that is formed after winding or 3D printing microtissues may then be matured in a bioreactor. Although, the approach discussed above may move much of the tissue maturation process to the beginning of the process rather than the end, final maturation of the tissue construct may be needed to grow the tissue construct and enable functionality and stability between the deposited microtissues. Bioprinting (e.g., winding the biological fiber) of pre-matured and pre-aligned cells may result in enhanced control over tissue alignment and vascularization. This may greatly decrease the complexity during final maturation of the tissue construct in a bioreactor.

FIG. 3B is a flow diagram of an example process for a method of 3D-printing a 3D-printed microenvironment in accordance with the present description. In the example of FIG. 3B, a system and/or operator aligns cells in a first direction to create pre-aligned microtissues (300). In one example, muscle cells may be matured using bioreactor signals so that the muscle cells are aligned in the first direction to form biological fiber with pre-aligned cells. For instance, a system may produce cells within a gel in a biological fiber. This biological fiber may include muscle cells which are not yet aligned. The biological fiber may then be spooled, under tension, to a spool structure. While the biological fiber is on the spool structure under tension, the cells remodel the hydrogel and may cause the gel to undergo compaction, inducing a strain in the hydrogel along the direction of the tension on the spool structure. The muscle cells can then align with their long axis parallel to the strain (or tension), resulting in highly aligned cells in the direction of the tension (e.g., which may be along the same direction as the length of the biological fiber). When released from the spool structure, the resulting biological fiber with aligned cells may be cut from the spool structure (or cut after removed from the spool structure) to form pre-aligned microtissues includes pre-aligned cells and may be added to a fluid or gel to form a bioink that is ready to be printed, or deposited, into desired shapes. In some examples, pre-aligned microtissues may be created to have widths or diameters from approximately 100 μm to approximately 200 μm, but smaller or larger widths may be used in other examples. In some examples, microtissues may be created to have lengths from approximately 5 mm to approximately 10 mm, but smaller or larger lengths may be used in other examples. In other examples, microtissues may be formed with pre-aligned cells using techniques other than strain between posts, such as using grooved (nanogroove) patterning of wells, electrical field alignment, or chemical alignment.

The pre-aligned microtissues can be combined (e.g., suspended) with a liquid (e.g., liquid or gel) to create a bioink (302). For instance, for aligned tissue bioprinting, pre-aligned microtissues (e.g., microtissues with a diameter of 250 μm and a length of 1 cm) may be aligned in the printing direction when printed as a suspension in GeIMA bioink.

A nozzle may then deposit the bioink laden with pre-aligned microtissues into a larger tissue construct of a desired orientation (e.g., a desired direction of alignment that may be different than the direction of alignment of cells within each microtissue) (304). For instance, rather than scaling-up the size of existing methods, pre-aligned and pre-vascularized microtissues may be produced and then used as “building blocks” in a 3D bioprinter to make larger tissue constructs. This approach may move much of the tissue maturation process to the beginning of the process (e.g., creating the microtissues used in the bioink) rather than the end (after all cells are printed in the desired shape of the tissue construct). This may greatly decrease the complexity during final maturation in a bioreactor. Bioprinting of pre-matured microtissues may result in enhanced control over tissue alignment and vascularization. In some examples, the nozzle may enable printing a 3D structure with the pre-aligned microtissues being deposited across two-dimensions and in height to create the 3D tissue construct. In other examples, the nozzle may be used to print multiple two-dimensional layers of microtissues, and then the different two-dimensional layers can be arranged and layered as desired to create a 3D larger tissue construct.

The larger tissue construct may then be matured in a bioreactor (306). Although, the approach discussed above may move much of the tissue maturation process to the beginning of the process rather than the end, final maturation of the tissue construct may be needed to grow the tissue construct and enable functionality and stability between the deposited microtissues. Bioprinting of pre-matured and pre-aligned microtissues may result in enhanced control over tissue alignment and vascularization. This may greatly decrease the complexity during final maturation of the tissue construct in a bioreactor.

Examples of the present disclosure demonstrate the ability to make the pre-aligned microtissues. It is possible that microtissues that have too small of a size may not maintain their alignment. A meso-scale method for printing as demonstrated above may be used. Microtissues may be printed into more complicated geometries with this meso-scale approach to printing. In traditional methods, during maturation of the printed tissues, the gSMCs show decreased proliferation if they are more of a synthetic phenotype in the microtissue. This may be addressed by supplementation of individually suspended gSMCs, that have high proliferative capacity and may “fill the gap” between microtissues. While it is possible the tissues may not be contractile enough to measure, this outcome is unlikely as gut SMCs ability to form contractile tissue in hydrogels has been demonstrated. Histological evaluations demonstrating alignment may be beneficial to other fields of tissue engineering as there is value in aligning non-contractile tissues as well. Maintaining the contractile phenotype may be beneficial.

Printing of a tissue with pre-vascularized microtissue bio-ink may result in faster development of capillary networks compared to traditional individually suspended cell bioprinting. While pre-vascularization of microtissues has been accomplished in the context of spheroids but has not been applied in 3D bioprinting smooth muscle. Capillary tube formation may be demonstrated using multiple stromal and endothelial cell types. In examples of the present disclosure, pre-vascularized spheroids using gSMCs and human umbilical vein endothelial cells (hUVECs) may be developed. HUVECs are a widely used model cell type for endothelial cell experiments, and readily form capillary networks in 3D culture. In addition, other cells could be developed from induced pluripotent stem cells (iPSCs) or other stem cell or progenitor cell populations to avoid immunologic rejection of engineered tissues upon implantation. In this manner, the cells may be derived from any human or mammal to create histocompatible tissues. Although pre-vascularized spheroids have been made previously, gSMCs have not yet been used.

FIGS. 4A, 4B, and 4C are example cross-section and profile views of example biological fibers generated using a core-shell spinning technique. One example process for microtissue formation involves a casting process. For example, hydrogels and cells can be cast into a mold (e.g., the biological fiber) and allowed to mature and contract to form the biological fiber that includes pre-aligned cells (or later cut into pre-aligned microtissues). This casting process can take more time and reduce throughput of pre-aligned cells in the biological fiber or pre-aligned microtissues compared to other production techniques. For example, a different method of production of the biological fiber may utilize a core-shell wet spinning approach. This process of co-axial or core-shell spinning can produce very long biological fibers made from cells 406 and a hydrogel 404, within shell 402. In this core-shell approach, cells 406 can proliferate and exert contractile force in hydrogel 404, which leads to alignment of cells 406 along the complete fiber direction (e.g., in the direction parallel with a longitudinal axis of the fiber). The material of shell 402 can be dissolved away after maturation, leaving only the core cellular fiber behind, which include cells 406 as part of pre-aligned microtissues.

The technique can use the wet spinning method to create long fibers which will then be used in that length for winding into a tissue construct or divided into smaller tissues and collected for use in bioprinting. For example, wet spinning can produce a continuous biological fiber which is then be cut into smaller pieces. In another example, these smaller pieces may be formed into a “pulsed fiber” as shown in FIG. 4C which would compartmentalize small pieces of cellular core material (e.g., cells 406 and hydrogel 404) into discrete microtissue units 420. Rather than cutting a long fiber into shorter fiber fragments to create the microtissues, a pulsed fiber only requires that the shell 402 be dissolved or digested away in order to expose the individual microtissue units 420 of that length for collection and printing.

In one example, the technique may use alginate as a shell material, which encases a core of cells and collagen or some other pre-gel solution. After maturation of the cells and collagen into a fiber with pre-aligned cells, the technique may involve dissolving the alginate shell material by chelation of calcium ions or by an alginate degrading enzyme such as alginase. Microtissues can be produced as short lengths (lengths between approximately 200-500 microns and length to width aspect ratios between approximately 2:1-5:1), allowing for high printing resolution and small curvature radius during the following bioprinting process. Microtissues can also be produced as longer segments (lengths between approximately 500-2000 microns) and a high aspect ratio (e.g., length to width aspect ratios of 5:1-20:1), which may enable better physical entanglement and strength of any tissues printed with these microtissues. This process may enable a system to generate discrete and repeatable microtissue units 420 in a very rapid manner.

FIGS. 5A and 5B are conceptual diagrams of an example spinning device 500 for wet spinning biological fibers or microtissue units 420 of the fiber of FIGS. 4A, 4B, and 4C. Spinning device 500 can control the size of the core fiber by controlling the flow rates of the material (e.g., shell material, cells, and hydrogel, through spinning device 500). The fiber units generated by spinning device 500 can be spooled onto a spooling device for maturing into the fiber with pre-aligned cells and eventual removing and wound onto a tissue frame to create a tissue construct or cutting and loading into a printer and deposited by the bioprinter in a non-continuous manner. Such a process may be used for many complicated structures like the heart, gastroesophageal junction, etc. For example, these small microtissues can later be assembled into larger tissues by bioprinting to exert much more control over the quality and character of the final product.

As shown in the example of FIG. 5A, spinning device 500 includes three different inlets, core stream inlet 502, shell stream inlet 504, and sheath stream inlet 508. Each of these inlets injects material to form a respective portion of biological fiber. Cells and the core material is injected first through core stream inlet 502. Then, the shell material is added through shell stream inlet 504 and added circumferentially around the core (e.g., at locations radially outward from the core) via spinneret 506. This combined material may be referred to as the biological fiber or units. Then, sheath material is added through sheath stream inlet 508 and added to the outside of the microtissue from spinneret 506 via spinneret 510 to create the output material, e.g., the biological fiber, that can mature into pre-aligned cells of the fiber that can be wound or cut up and printed.

In this example, spinning device 500 can use a series of coaxial nozzles or spinnerets (e.g., spinnerets 506 and 510) to layer the shell material over the core that includes the cells to be pre-aligned within the shell. For example, spinneret 506 can inject a stream of core fluid into the center of a larger stream of shell fluid. Then, the coaxial fluid streams are injected through a second spinneret 510 into the center of a larger stream of fluid (e.g., the sheath material) which can contain crosslinking chemicals to convert the coaxial core and shell streams into a solid biological fiber. In another example, all three streams can be introduced at the same point by a coaxial nozzle made of three independent nozzles of decreasing size inserted into each other.

The example of FIG. 5B illustrates a conceptual diagram of spinning device 500. As shown, core stream inlet 502, shell stream inlet 504, and sheath stream inlet 508 can converge within a fluid manifold that may take the form as shown in FIG. 5A or other fluid flow channels. Outlet material 530 is shown as a cross-sectional view of the coaxial material and includes core stream 520 in the middle, shell stream 522 radially outward from core stream 520, and sheath stream 524 radially outward from shell stream 522. In this manner, shell stream 522 may depict the shell of the spun fiber, which can provide structural support during development or pre-aligning of the cells within the fiber. Core stream 520 includes the core of the biological fiber, which contains cells and a hydrogel material that develop into the pre-aligned cells of the biological fiber.

The flow rate of material out of spinning device 500 may be varied based on cell size, hydrogel viscosity, or other factors. In one example, the flow rate may be between approximately 20 microliters per minute and 200 micro liters per minute. In another example, the flow rate may be between approximately 50 microliters per minute and 100 microliters per minute. In an example at 50 microliters per minute, the shell stream 524 may have a diameter of approximately 285 micrometers, and core stream 522 may have a diameter of approximately 94 micrometers. In another example at 100 microliters per minute, the shell stream 524 may have a diameter of approximately 35 micrometers, and core stream 522 may have a diameter of approximately 173 micrometers. These example dimensions are merely for illustrative purposes, as other flow rates and dimensions are possible by using different characteristics of the material and spinner device 500.

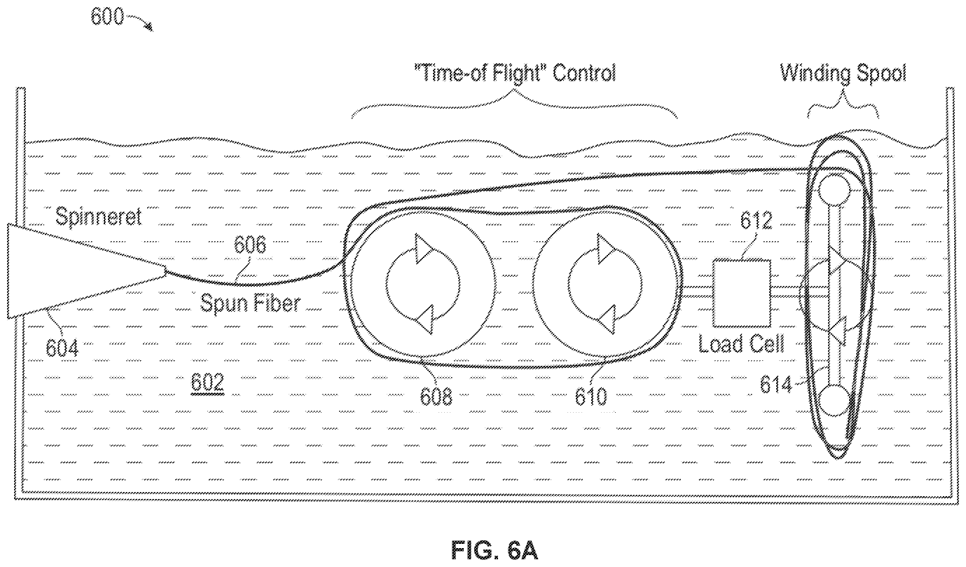

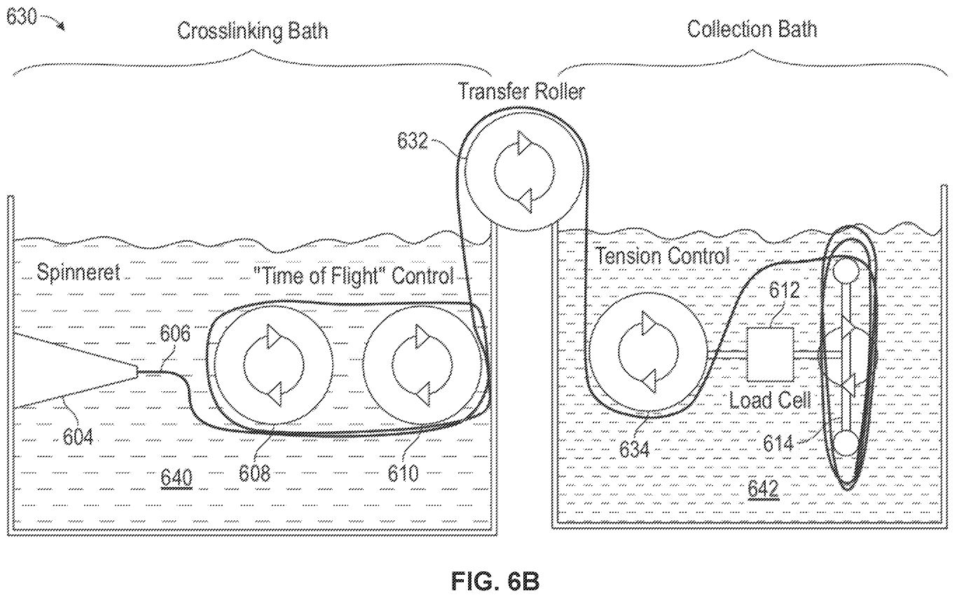

FIGS. 6-10 describe techniques and systems for spooling the biological fiber onto a spool structure for further maturing the fiber into the biological fiber that includes pre-aligned cells (and may be cut into pre-aligned microtissues). FIG. 6A is a conceptual diagram of an example system 600 for spooling biological fibers in a crosslinking bath. As shown in FIG. 6A, the spooling of the biological fiber 606 take place in a moist or submerged (e.g., within a solution) environment 602. The solution 602 may be a crosslinking bath that supports the biological fiber 606. The system 600 collects the biological fiber 606 dispensed into the solution 602 from spinneret 604 (which may be similar to spinnerets 506 or 510) onto one or more rollers 608 and 610. These rollers 608 and 610 may enable the fiber 606 to remain within the solution 602, such as being exposed to crosslinking agents of the solution 602, for a target amount of time. Although two rollers 608 and 610 are shown, a single roller, or three or more rollers, may be used in other examples. Rollers may have the same diameter or different diameters in other examples. This is the “time of flight” control to the fiber 606. After the one or more rollers 608 and 610, the winding spool (e.g., spool structure 614) spools the biological fiber 606 for storing and maturing into the biological fiber that will contain pre-aligned cells in the direction of the tension. The load cell 612 coupled to the spool structure 614 may measure the tension applied to the biological fiber 606 as it is spooled onto the spool structure 614. The system 600 may then control the rotational speed and/or position of the spool structure 614 with respect to the rollers 608 and 610 in order to maintain a target tension of the biological fiber 606 during the spooling process. The spool structure 614 is shown as a “fork” structure that includes two parallel prongs (extending into the page as shown) around which the biological fiber 606 is wound. Any spool structure 614 may be used, such as a cylindrical drum, ovular drum, or forks with three or more prongs. In some examples, the spool structure 614 may include a surface configured to promote friction with the biological fiber 606 that prevents the fiber from moving on the surface of the spool structure 614. In other examples, the spool structure 614 may have a low coefficient of friction configured to promote the biological fiber 606 slipping or moving on the surface in order to allow the tension in the biological fiber 606 to even out along the fiber.

FIG. 6B is a conceptual diagram of an example system 630 for spooling biological fibers in a collection bath separate from a crosslinking bath. The system 630 of FIG. 6B may be substantially similar to the system 600 of FIG. 6A. However, the system 630 of FIG. 6B may include multiple solution baths 640 and 642 during the process of spooling. There may be scenarios in which a crosslinking bath may be detrimental to the cells within the biological fiber 606. Therefore, multiple baths may enable the system to separate the process and provide the flexibility to prevent over-exposure of the fiber to the crosslinking agents in these cases. In the example of FIG. 6B, spinneret 604 produces biological fiber 606 which is then captured by one or more rollers 608 and 610 as described above with respect to FIG. 6A. Then, biological fiber 606 is taken up by transfer roller 632 which passes the biological fiber 606 from the crosslinking bath 640 to the collection bath 642 with a different solution than crosslinking bath 640. Another roller 634 in the collection bath 642 may be coupled to a motor (not shown) that controls rotation and/or position of the roller 634 in order to maintain a target tension on the biological fiber 606 as it is spooled on the spool structure 614. As discussed in FIG. 6A, the load cell 612 may measure the tension applied to the biological fiber 614 during the spooling of biological fiber 614 onto spool structure 614.

Control over the tension during the spooling process may be important to one or more aspects of processing and or biological efficacy. From a manufacturing perspective, the system 600 or 630 may exert enough tension that the spooling occurs on the spool structure 614, but not too much tension that the fiber breaks under the applied load. The control system (not shown) may also manage the rotation of the spool structure 614 and/or other rollers in the system order to coordinate the spooling speed with the biological fiber 606 dispensing speed at the spinneret 604. The control system may include one or more controllers (e.g., processing circuitry or other circuits) configured to receive sensed data from load cell 612 and/or other sensors and then control the speed of one or more motors coupled to one or more rollers and/or spool structure 614 to maintain the desired tension on biological fiber 606.

The Time-of-flight control refers to the time that a unit length of biological fiber 606 is subjected to the crosslinking bath 640. Depending on the crosslinking method, different amounts of time may be used for desired development of the biological fiber 606. This can be accomplished by running the fiber 606 multiple times around a set of rollers 608 and 610. An increased number of wraps around the rollers leads to an increased crosslinking time before either spooling or transferring the biological fiber 606 to another bath for spooling on spool structure 614. In either case, the biological fiber 606 may be developed enough using the crosslinking bath to acquire the strength to withstand the spooling and/or transfer process into the spool structure 614.

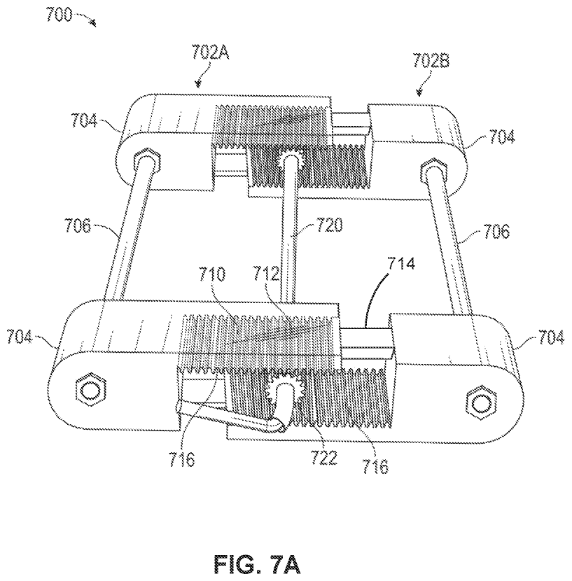

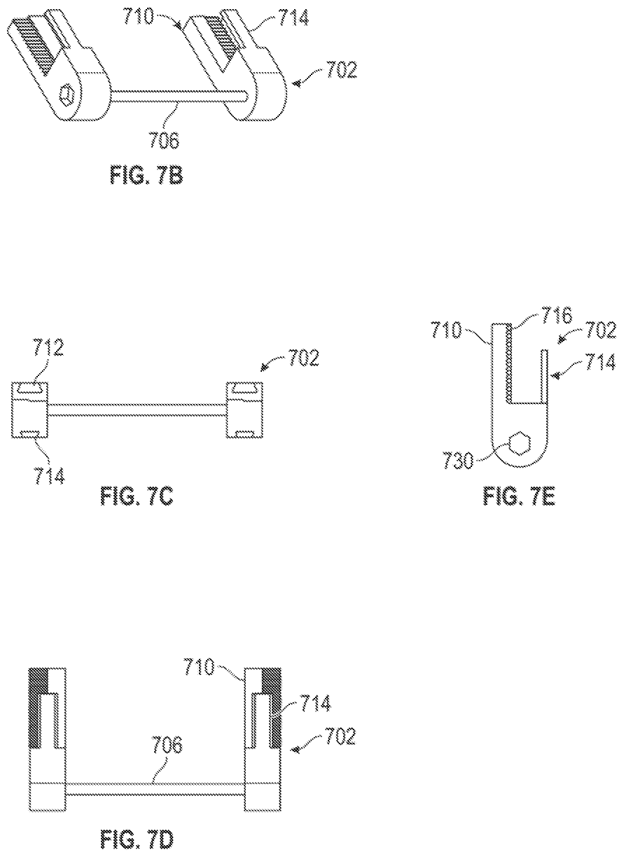

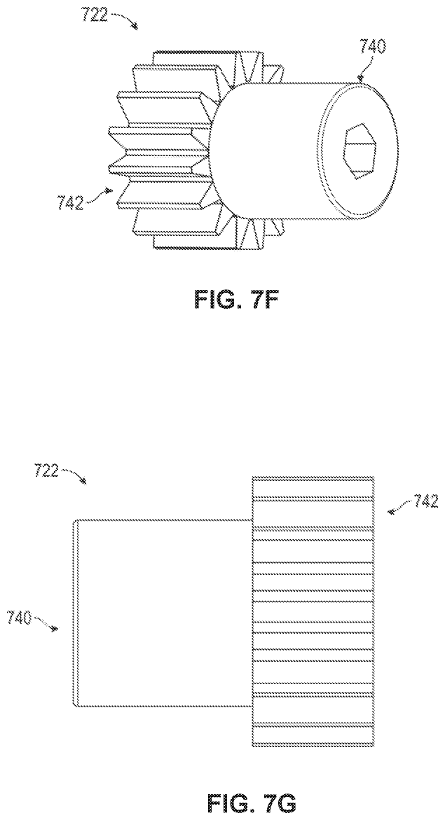

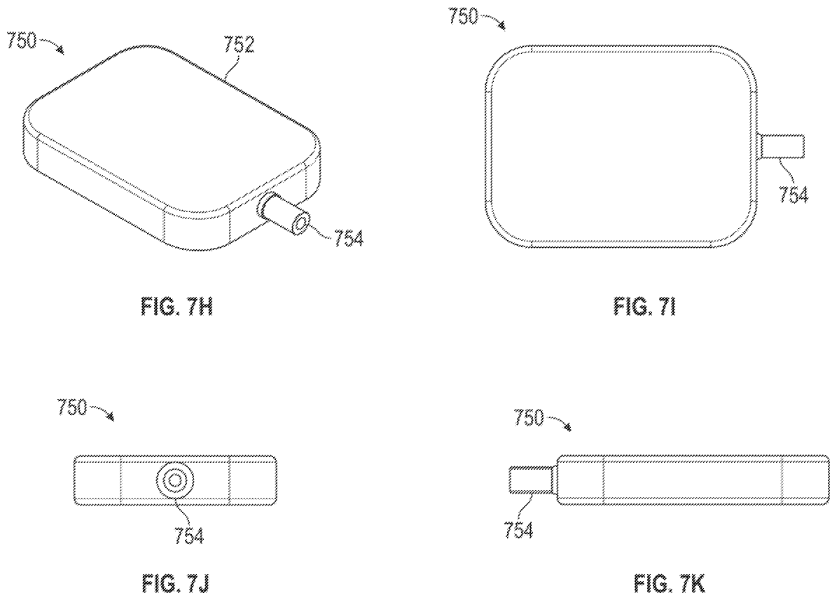

FIG. 7A is a conceptual diagram of an example expanding device for creating tension on biological fibers. As shown in the example of FIG. 7A, expanding device 700 is an example spool structure that is configured to increase the dimension between bars 706. Biological fiber may be spooled onto bars 706. Each of bars 706 is connected to a pair of arms 704. One pair of arms 704 and one bar 706 makes up one movable structure 702A or 702B. Together, two movable structures 702A and 702B can move in one dimension with respect to each other.

For stability, each arm 704 includes rack 710 with teeth 716 on one side of rack 710. Rack 710 also defines a slot 712 within a distal end of rack 710. Each arm 704 also includes protrusion 714 which is configured to mate to slot 712. In this manner, each protrusion 714 can slide within a respective slot 712 of the adjacent rack to allow for movement in one dimension and stability in other dimensions. A pinion gear 722 is located on axle 720 and configured to mate to the teeth of opposing racks of each arm. Rotation of axle 720 will cause pinion gears 722 to rotate and translate the rotational movement to linear movement between the racks of each arm. This linear movement in either direction of the one dimension can cause the distance between bars 706 to increase or decrease. In this manner, rotation of axle 720 can increase or decrease the tension on the biological fiber spooled on expanding device 700 by changing the distance between bars 706.

FIGS. 7B, 7C, 7D, and 7E are different views of the expanding device 700 of FIG. 7A. FIG. 7B shows a perspective view of movable structure 702A (which is the same as movable structure 702B). Both rack 712 and protrusion 714 extend in the same direction in a parallel manner to provide sliding with respect to an opposing movable structure 702B.

FIG. 7C shows a front view of movable structure 702 to show the channel of slow 712 and the axis of protrusion 714. FIG. 7D shows a top view of movable structure 702 which illustrates that the teeth of rack 710 are only on the outside of rack 710. In other examples, the teeth may be provided on the center, inside, or entire surface of rack 710. FIG. 7E shows a side of view movable structure 702 that illustrates teeth 716 on a surface of rack 710 and facing protrusion 714. Each arm 704 defines an opening 730 that accepts an end of bar 706. In other examples, bar 706 may be attached to the arm without an opening.

FIGS. 7F and 7G are different views of pinion gear 722 of expanding device 700 of FIG. 7A. As shown in the perspective view of FIG. 7F, pinion gear 722 includes a shaft 740 and teeth 742 extending radially out from shaft 740. FIG. 7G shows a side view of pinion gear 722.

FIGS. 7H, 7I, 7J, and 7K are different views of an inflation device 750 for creating tension on biological fibers. Inflation device 750 may be placed within a spool structure, such as spool structure 614 or other spool structure in order to add tension to biological fiber spooled on the spool structure. Inflation device 750 may be constructed of a flexible material such as a polymer that can be expanded by increasing a pressure of liquid or gas contained within inflation device 740. The exterior surface of inflation device 750 can then contact the biological fiber to increase tension on the biological fiber when spooled.

As shown in FIG. 7H, inflation device 750 may be constructed with an exterior surface 752. In some examples, two large flat opposing surfaces of exterior surface 752 may be flexible while the narrow edges are rigid. In other examples, the entire exterior surface 752 may be flexible and expand with increased internal pressure. Inlet 754 may be attached to a hose or other structure to add or remove fluid and/or gas from within inflation device 750. FIG. 7I is a top view of inflation device 750 that has rounded corners. FIGS. 7J and 7K are side and end views of inflation device 750. Although inflation device 750 is shown as a rectangular structure, inflation device 750 may be constructed of any shape, such as a cylinder, sphere, or other three dimensional structure configured to fit within the biological fibers spooled on a spool structure.

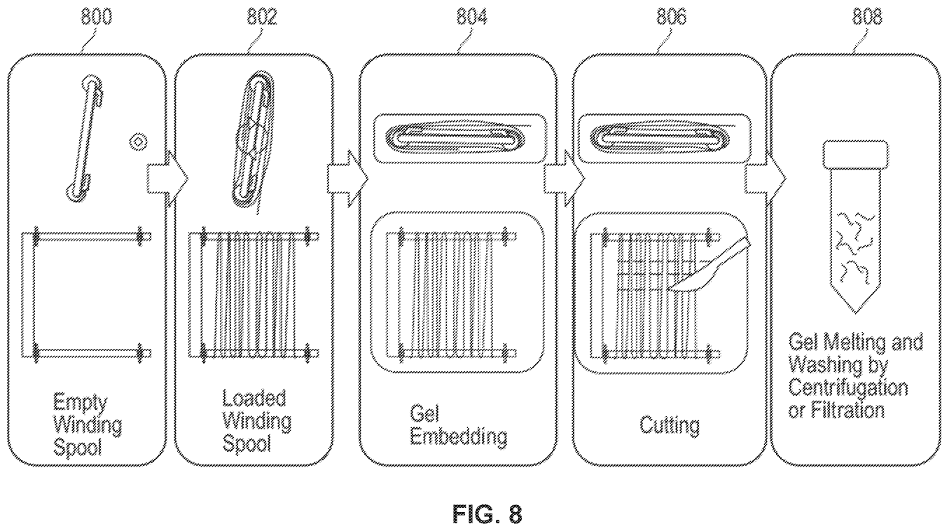

FIG. 8 is a flow diagram of one example of cutting biological fibers from a loaded spool to create pre-aligned microtissues for use in a bioink and eventual 3D printing. In the example of a spool structure like a two-pronged fork, the system can start with an empty winding spool (800) and then load the winding spool with a biological fiber as described herein (802). A two-pronged fork may be configured such that it can sit flat inside of a petri dish or similar shallow vessel. While submerged, these biological fibers tend to be difficult to cut because they are slippery and are free to move back and forth with the fluid. To prevent this problem the spool and attached fiber can be embedded into a reversible gel (804). In one case, this gel can be made of gelatin, which is thermoreversible. The biological fibers are put into a dish and the liquid gelatin is poured into the dish. The dish is then cooled briefly to solidify the gel. While the gel is solidified, the biological fiber can be cut while the fibers are locked inside of the solidified gel (806). The gel is then melted away (808), leaving the cut fibers behind. When paired with robotic cutting or cutting molds, this can yield uniform fiber lengths.

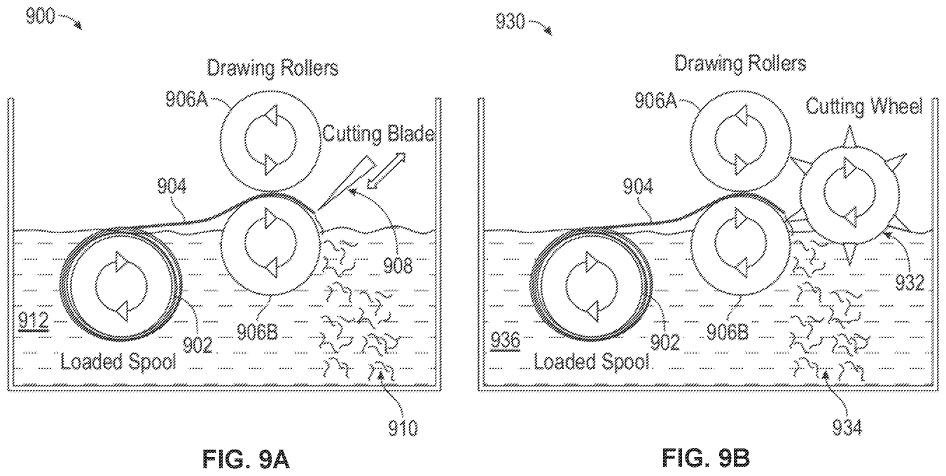

FIGS. 9A and 9B are conceptual diagrams of example systems 900 and 930 for cutting biological fibers 904 from a loaded spool 902. Another method for creating uniform fragments from the biological fiber 904 after it includes pre-aligned cells is in-line cutting. This could be done as the fiber is being produced, but it is more likely that it will be done after the fiber has been matured for several days to allow the cells to form a stable pre-aligned microtissue. In one example, the fiber has been matured for several days and is loaded on a spool structure 902. This spool 902 can then be unwound through a series of one or more drawing rollers 906A and 906B (collectively “drawing rollers 906”) and one or more cutting devices, such as butting blade 908 or cutting wheel 932. Compared to gel embedding. this method of cutting may be more automated and take less processing time. The cutting may be done just above the surface of the bath fluid, so that the fiber is stuck to the cutting rollers by fluid forces when in the air for clean cutting. However, the fiber fragment is then resubmerged, releasing the surface tension and allowing the fiber to fall to the bottom of the bath chamber for collection.

As shown in FIG. 9A, cutting the biological fiber 904 from the drawing rollers 906 may include moving a cutting blade 908 linearly (e.g., toward and away from the fiber) at a frequency that cuts the fiber 904 in a target length according to the fiber distance moved by the roller. As shown in FIG. 9B, cutting the biological fiber 904 from the drawing rollers 906 may include passing the fiber 904 by a roller (e.g., cutting wheel 932) with one or more cutting blades that cut the fiber 904 as each blade rotates toward the fiber 904. In other examples, a system may include one or more cutting blades that move laterally across the location of the fiber in order to cut the spooled fiber into target lengths.

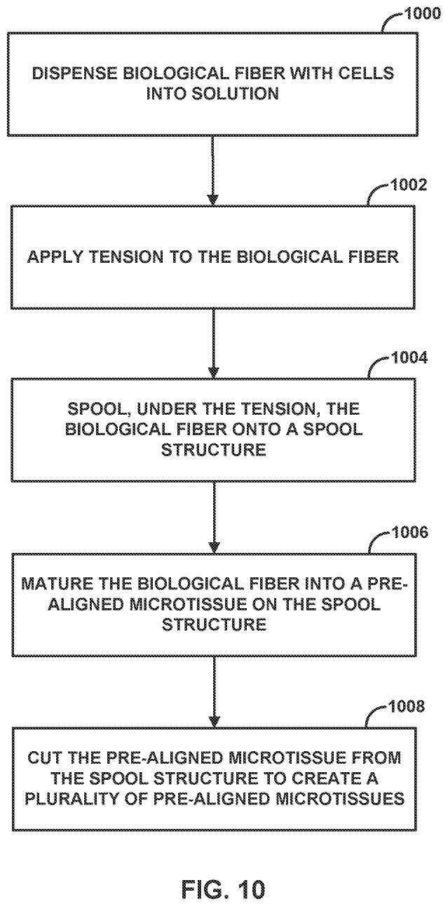

FIG. 10 is flow diagram of an example technique for spooling biological fiber and then cutting the spooled biological fiber to specific lengths to form pre-aligned microtissues. The flow diagram of FIG. 10 may use systems such as systems 600, 630, 900, and/or 930. As shown in the example of FIG. 10, the method may include dispensing a biological fiber into a solution, wherein the biological fiber comprises a plurality of cells within a hydrogel (1000). The solution may be a crosslinking bath or other fluid. The dispensing step may be performed by a spinneret or other structure that forms the biological fiber from cells and a gel, for example.

The system may then apply tension to the biological fiber (1002). The tension may be applied by the speed of one or more rollers or spool structures provided by one or more motors. In some examples, the system may control the speed of the rollers or spool structures by measuring the tension and adjusting the speed in order to achieve the target tension. In this manner, this process may be closed-loop and include applying the tension may include measuring the tension applied to the biological fiber during the spooling and controlling, based on the tension measurement, a speed of the spooling that applies the tension to the biological fiber.

The system may then spool, under the tension, the biological fiber from the solution and onto a spool structure (1004). In one example, spooling the biological fiber may include wrapping the biological fiber around the spool structure within a fluid comprising the biological fiber. In this manner, the spool structure may be submerged within the solution during the spooling of the fiber onto the spool structure. In another example, spooling the biological fiber may include wrapping the biological fiber around the spool partially submerged in a fluid or completely separate from the fluid, the fluid comprising the biological fiber. In this manner, the spool structure may be only partially submerged or completely out of the solution. The system may apply the solution, or a different fluid, to the spool and/or fiber on the spool to maintain wetness of the fiber and viability of the cells during the spooling process.

While on the spool structure, the system may mature the biological fiber into a pre-aligned microtissue on the spool structure (1006). For example, the spool structure may be placed within a bioreactor so that the cells may stay alive while subjected to the tension applied to the fiber when collected by the spool structure. In this manner, the tension applied to the biological fiber on the spool structure at least partially aligns the plurality of cells in a direction of the tension to form a pre-aligned microtissue from the biological fiber. In some examples, the spool structure may be an expanding structure (e.g., expanding structure 700) to increase the tension as desired or include a separate expanding structure for adding tension to the biological fiber.

Once the biological fiber has been matured, the technique may include cutting the biological fiber with pre-aligned cells from the spool structure into a plurality of pre-aligned microtissues (1008). The pre-aligned microtissues have a length to width ratio of at least 3:1 in some examples, in one example, the cutting process may include embedding the spool structure with the biological fiber into a gel, cutting the gel and embedded spooled biological fiber into a plurality of pre-aligned microtissues, and removing the plurality of pre-aligned microtissues from the gel by melting the gel from the plurality of pre-aligned microtissues.

In another example, the cutting process may include unspooling, from the spool structure, the biological fiber to a drawing roller and cutting the biological fiber on the drawing roller into a plurality of pre-aligned microtissues. In any case, the resulting pre-aligned microtissues may be used for printing a tissue construct. For example, the printing process may include suspending the pre-aligned microtissues in a liquid to create a bioink and depositing the pre-aligned microtissues in a second direction to create the tissue construct.

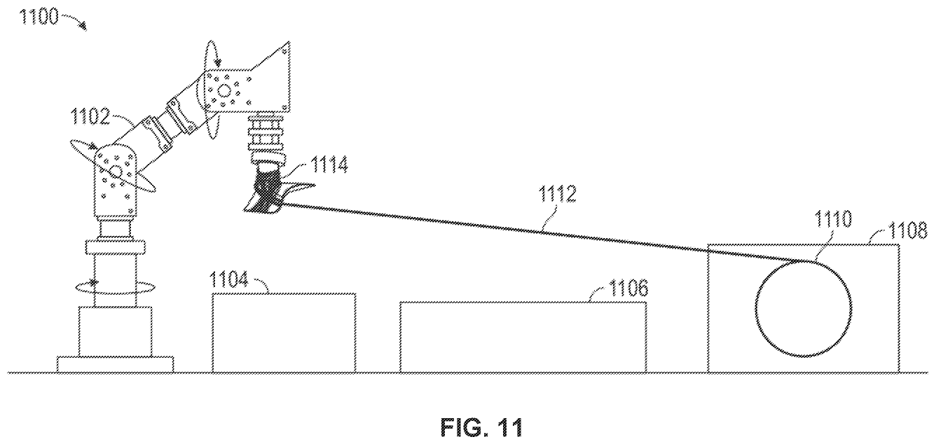

FIGS. 11-18 describe techniques and systems for winding the biological fiber onto a tissue frame for generating a tissue construct (e.g., an organ or other tissue) with cell fibers oriented in the direction of the fiber wound on the tissue frame. The biological fiber may have been spooled onto a spool structure as described herein in order to store and/or provide tension to the biological fiber which causes the cells to pre-align in the direction of the tension. In some examples, tension is again applied to the biological fiber as it is wound onto the tissue frame. Tension may further be applied or increased to the biological fiber when on the tissue frame by expanding the tissue frame in one or more dimensions after the winding process. FIG. 11 is a conceptual diagram of an example system 1100 for winding biological fibers around a tissue frame to create a tissue construct.

Amongst the plethora of types that the robots can offer, the robot arm 1102 can deliver a certain number of degrees of freedom that can be utilized in any application. Robot arms are also very versatile and can provide a number of functions with minimal control. These arms are also quite precise and can be optimized for almost any function. Moreover, robot arms are available in many shapes and sizes based on the application. Robotic arms can also be programmed to offer various ways to control thus giving users a choice on how to generate the tissue construct. Robotic arms are also one of the easiest to simulate and troubleshoot potential issues at a very early stage As shown in the example of FIG. 11, the system 1100 may include a robotic arm 1102 with multiple degrees of freedom, such as 6 DOF in some examples. The biological fiber 1112 may be contained on a spool structure 1110 in a liquid bath 1104 to support hydration of the cells in the biological fiber. The robotic arm 1102 may have an end effector that is attached to a tissue frame 1114. An end of the biological fiber 1112 (e.g., a muscle fiber) may be attached to a portion of the tissue frame 1114. Then, the robotic arm 1102 can move in a predetermined spatial path that pulls the biological fiber 1112 from a collector mechanism (e.g., a spool structure 1110) and lays down the biological fiber 1112 onto the tissue frame 1114 in a direction determined from the spatial movement of the tissue frame 1114 controlled by the robotic arm 1102. The robotic arm 1102 may use sensor feedback, such as speed data, tension data, etc., to control the speed of any motors at each axis, the speed of the end of the robotic arm, and/or the speed of the biological fiber 1112 pulled from the spool structure 1110.

A robotic arm 1102 can have the capability to move an object in various axes as well as with minimal modifications the application can be robust and can be repeatable. Coding a robotic arm for this application is also practical and can be done for the exact movements with utmost precision for the winding process. The following are various advantages of a robotic arm to perform the winding process for a biological fiber. The control and operation of the robot arm is very easy and modifiable. The robotic arm can perform the task accurately with a lot of changeable parameters based on the size and type of tissue or scaffold used. A robot is relatively inexpensive, can perform all the necessary tasks, can be developed with minimal hardware. Additional set up for adding ways to wind the fibers and to spool-unspool is also possible without any entanglement and pre testing for that is also very practical. Due to the compactness, a robotic arm is also repeatable at any scale and the integration of the whole system can be carried out seamlessly.

While the robotic arm is at the core of the winding system, there are multiple factors that can be considered for the running of the robotic arm for the winding process. The multi-axis winder created with the robotic arm also has external parts that help in the smooth working of the entire device. Following constraints may be used in some examples:
1. The device can be configured to wind the desired tissue, scaffold or organ in any orientation and on any axis required.
2. The device can be configured to move at variable speeds and also should be able to be controlled extremally.
3. The device can be configured to work with various tissues and scaffolds and should be able to switch and modify for each of the applications.
4. The device can be configured to work with a spooling system and a bridging system between these two components.
5. This device can be configured to be compact yet modifiable for various sizes and shapes.
6. This device can be configured to collect data from its winding motions so that a universal standard for such a device can be set
7. This device can be configured to work well with moisture and can be able to keep the cell fibers moist during the winding process.
8. The device can be configured to produce continuous winding and there should not be any need for the cell fiber to be cut and attached multiple times.
9. The device can be configured to be controlled with respect to the parameters such as number of rotations, size of the scaffold, speed of rotations, etc. In short, the device should be automated but should allow for manual override.

Of all the components used in the construction of this device, the robot arm plays an important role. There were many parameters that needed to be considered for the selection of the robot arm. The first factor used was the size of the robot which ideally should be varying according to the scaffold or tissue that needs to be wound. Based on the fact that most of the tissues or scaffolds that need to be wound using this device are between the size range of 2 cm to 6 cm, a tabletop or a desktop-sized robot arm is a viable option. Most robots in this size can provide various flexibility, such as 4 DoF Robots, 5 DoF Robots, or 6 DoF Robots. Degrees of freedom refer to the number of movable linkages in the robot that can be used for the application. This robot arm may need to move in multiple axes and hence having more degrees of freedom can be certainly beneficial, which leads to 6 DoF robot arms. Although 6 DoF arms are described herein, some winding techniques may be used with robot arms having fewer degrees of freedom.

Servo motors are very dependable, easy to program and a highly controlled type of motors that give full flexibility for such an application where the angles of rotation matter the most. The 240 degrees max rotation and 270 degrees max rotation range of servos can be suitable and carry out the desired function for the robot arm. Each motor for each rotational axes of the robot may have the same or different rotational maximum per the function of that axis. In other examples, full 360 degree rotational motors may be used, but may be more difficult to control if they are not position controlled. Precise rotational control may be helpful to reduce the likelihood of entanglements when the cell fibers are introduced.

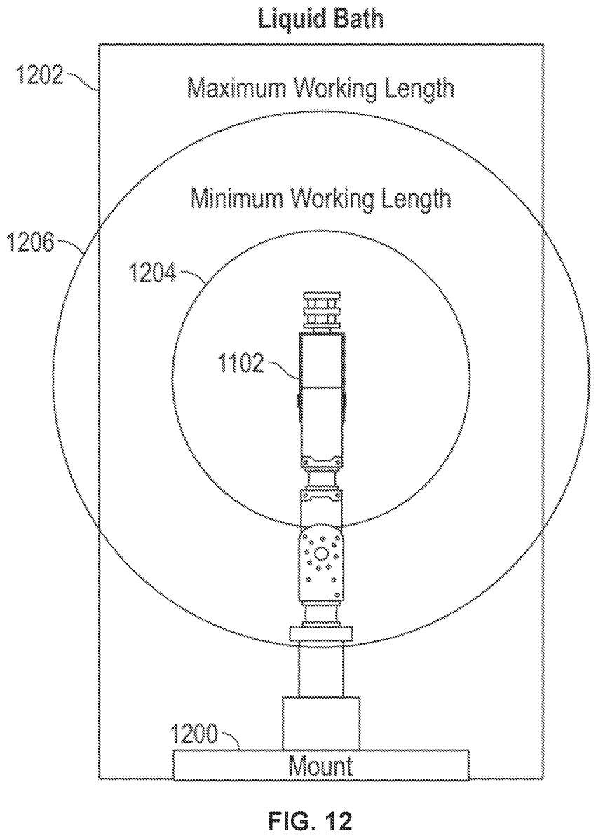

A factor for the determining of any robot arm is the working envelope, as shown in FIG. 12. This term defines the full working radius of the robot in all directions. This work envelope is also determined with the size of the linkages; which may have linkage sizes between 2 inches and 4 inches for some tissue constructs. These lengths can be selected based on the largest tissue or scaffold that is required to be wrapped, which could be an adult GEJ having length of 3 to 4 cm. Other linkages of the robot arm may be selected in other examples. The robot arm also includes an end effector that may be customizable to enable different ways to connect the tissue frames or other elements related to the process for the winding action to be performed.

This robot arm may be controlled using any of the following example micro-controllers: Arduino, Raspberry Pi, or Jetson Nano. All of these microcontrollers can be programmed in multiple languages. In one example, for an easy yet robust controllability of the robot, the ARM controller, and the Arduino microcontroller was selected with python and GUI based programming as the primary coding languages.

In one example, the system may include a robotic arm with the following characteristics:
Size: 154×140×246 mm
Body weight: about 0.9 kg
Material: aluminum alloy and surface sandblasting and oxidation
Power supply: 7.5V 6A DC power adapter
Controller system: xArm 1S bus servo controller
Software: PC software and mobile APP
Servo: single shaft/LX-15D/LX-225 intelligent bus servo
Control method: iOS/Android mobile APP/PS2/mouse/PC software

FIG. 12 is a conceptual diagram of distance restraints for an example robotic arm 1102. As shown in the example of FIG. 12, a top view diagram illustrates the working envelopes with the dot being the working center (point at which the cell fiber is attached to the organ frame). Path planning or mapping (e.g., the predetermined spatial path) for the tissue frame attached to the robotic arm enables the robotic arm to wind any tissue or scaffold. Circle 1204 represents the minimum working length of robotic arm 1102, and circle 1206 represents the maximum working length of robotic arm 1102. Robotic arm 1102 may be attached to mount 1200 and be configured to work within liquid bath 1202.

The first part of this process to generate the spatial path may be to physically move the robot joints to check various patterns and orientations. The first pattern developed after the initial phase of testing can be made for the multi-axis tool that demonstrates the switching from one axis to another and then a continuous rotation. Then, the path mapping can be completed for the tissue construct, such as the GEJ.

The second step can be to understand all the various movements and rotations the robot arm can perform. This could be done by creating a simulation of this robot arm using ROS. For simulating the robot, a repository was already available which could be used to move various joints of the robot in all the combinations possible. This simulation can enable understanding the full range work envelope of the robot as well as to what extent the motors can move the end effector without entanglement. The simulation also laid the foundation to choose movements that look promising for winding the biological fiber at varying axes on the surface of the tissue or scaffold.

The method used to decide the trajectory for the robot arm to follow for winding the tissue frame (e.g., an organ frame) can include the method of learning from demonstration. In this method, the approximate angles obtained from the simulation as well as from manually moving the robot arm to various locations and through various trajectories, can be input in the algorithm further described. These angles can then be changed in gradual increments or decrements based on the servo motors used and a close to optimum trajectory was developed. In the example of a tissue frame (or organ frame) for the GEJ, 3 different kinds of trajectories can be used. Each of these trajectories were used to wind one type of cell fiber winding.

Winding 1 (LECS): This type of winding has the simplest trajectory completed in 24 steps. Each step here is an action which involves movement of the robot linkages using the servo motor. 24 such actions can be used to make one rotation of the cell fiber around the tissue frame. This trajectory is selected to have a maximum work radius of 6 inches and for an optimal wind the minimum deviation from the home position is 2.5 inches,

Winding 2 (Clasp): This type of winding has the simplest trajectory completed in 24 steps. Each step here is an action which involves movement of the robot linkages using the servo motor. 24 such actions can be used to make one rotation of the cell fiber around the tissue frame. This trajectory is selected to have a maximum work radius of 6 inches and for an optimal wind the minimum deviation from the home position is 2 inches.

Winding 3 (Sling): This type of winding has the simplest trajectory completed in 24 steps. Each step here is an action which involves movement of the robot linkages using the servo motor. 24 such actions can be used to make one rotation of the cell fiber around the organ frame. This trajectory is selected to have a maximum work radius of 7 inches and for an optimal wind the minimum deviation from the home position is 3 inches.

Apart from these windings, the increment or the decrement of the angles of the servos were based on the angles obtained from the simulation but optimized to reduce the working envelope to a radius length well within reason and to minimize the amounts of action sets necessary to complete one rotation.

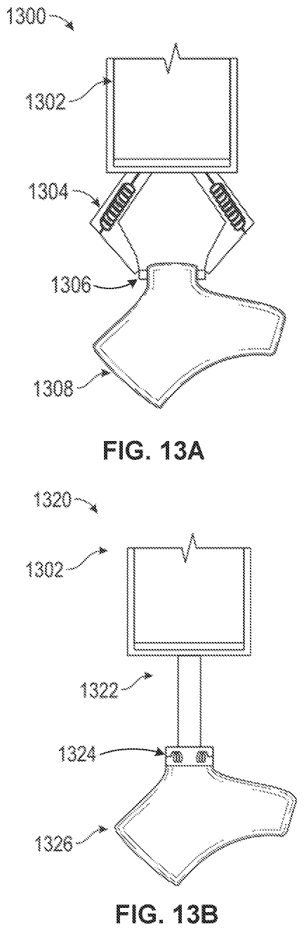

FIGS. 13A and 13B are conceptual diagrams of example connections from a robotic arm to a tissue frame. As shown in the example of FIG. 13A, the end link 1302 of the robotic arm of system 1300 can be attached to a 3D pointed grabber 1304 with springs that is configured to grip an edge 1306 of the tissue frame 1308. In the example of FIG. 13B, the robotic arm end link 1302 of system 1320 may include a post 1322 to which an expanding disk 1324 using springs can be attached. Tissue frame 1326 may be attached to post 1322 via interface with expanding disk 1324 that fits within a portion of tissue frame 1326. These are just some examples of methods for attaching the tissue frame to the robotic arm for the winding process.

In some examples, a tissue frame may be configured to expand in one or more dimensions to increase tension in the biological fiber that has been wound around the tissue frame. In this manner, when the biological fiber is wound around the tissue frame, tension on the biological fiber can be maintained or even increased to further aid in cell alignment in the direction of the tension and in the direction of the longitudinal axis of the biological fiber. In some examples, the tissue frame can be formed of a flexible material (e.g., polymer or flexible metal alloy) that can expand in volume by internal fluid pressure (e.g., liquid or gas) or internal pistons or other devices that push out on one or more surfaces of the tissue frame. Internal pressure within the tissue frame may be due to increase fluid pressure when tissue frame is a sealed volume that has an inlet for adding or removing fluid. In other examples, a tissue frame may be formed from multiple rigid sections that can be moved apart from each other by a mechanical screw drive or inflatable internal mechanism within the tissue frame.

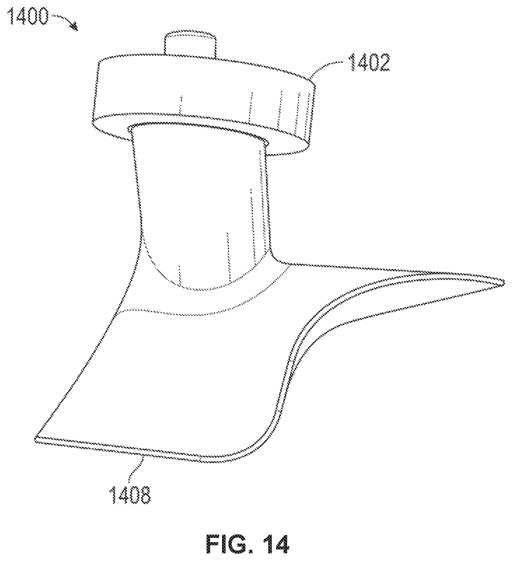

FIG. 14 is photograph of an example tissue frame 1400 for creating a tissue construct of a GEJ. As shown in the example of FIG. 14, a replica of the Gastro-esophageal Junction indicates the complexity of some tissue constructs that can be. The system may attach this tissue frame 1400 of the GEJ to the robotic arm and wind biological fibers around the frame to form the tissue construct. Example dimensions and material: 2×2×1.5 inches made of surgical guide resin. Tissue frames may be constructed of polymers or biodegradable materials. In some examples, the tissue frames may be sprayed or otherwise applied with a substance that promotes adherence of the fiber to the frame. For example, the substance may include growth factors or a biocompatible adhesive. Tissue frame 1400 may include a fiber surface 1408 for collecting the wound biological fiber and an attachment structure 1402 that is configured to be attached to or grabbed by the robotic arm.

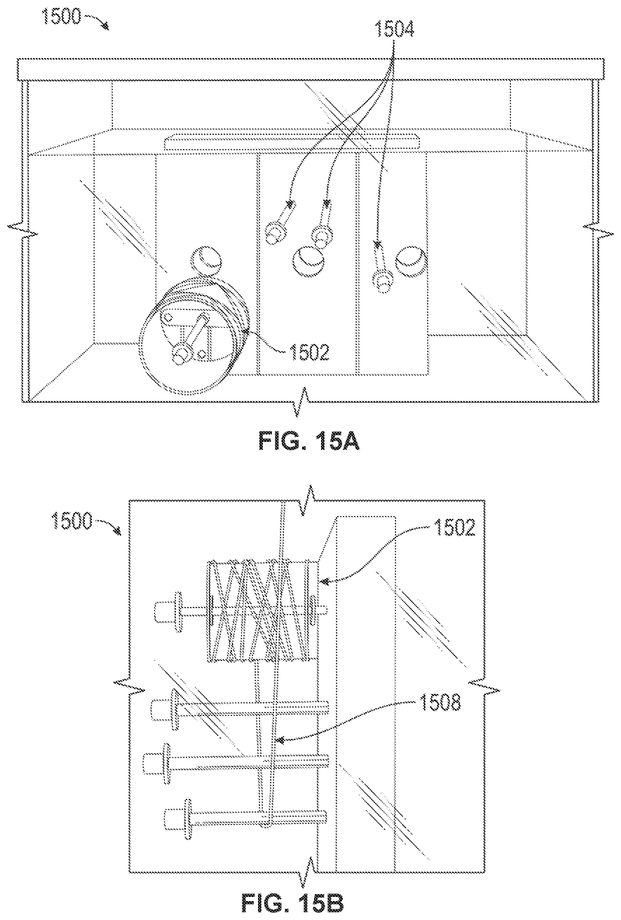

FIGS. 15A and 15B are front and side views of an example spool structure 1502 and bath 1500 from which a robotic arm winds the biological fiber. As shown in the example of FIG. 15A, the liquid bath 1500 may include the spool structure 1502 containing the biological fiber 1508 wrapped around it. Additional guides 1504 may enable the biological fiber 1508 to be threaded around one or more of the guides and to a position from which the biological fiber can be pulled by the robotic arm. FIG. 15B illustrates a side view of the bath and possible route for the biological fiber 1508.

As described herein, the system can be configured to wind the biological fiber around a tissue frame with some tension. This tension can further aid in maintaining or improving cell alignment within the biological fiber in the direction of the tension and in the direction of the longitudinal axis of the biological fiber. In some examples, the system may include a regulated tension control system. Any sort of system that involves threads or linkages (in this case biological fiber) may benefit from a system that can keep the fiber under tension at all times or at least when desired. For this application, the cell fibers can be in the tension so that they can be tightly wound around the required scaffold or tissue frame. In some examples, it may be desired to have minimum electronic isolation between the spool structure and the robot arm. This can reduce the number of coordination steps between the robotic arm and spool structure. As shown, a gravity-based tension system can be used.

This system can include 4 guide bars located at a calculated distance from one another. These bars are mounted on a panel of the dimensions 8×0.5×6 inches. This tension system uses the principles of weighted pulley system that introduces a dead weight in the system that helps the fiber between the robot arm and the last guide bar to be tightened at all times for winding motions. In this example, the spool having the collected biological fiber is attached to the first bar which is located at a height of 1 inch from the base of the mounting panel. Further, the biological fiber would be taken from the spool and would be draped around the first bar and then onto the second guide bar following the third and onto the robot arm. To control the movement of the cell linkage while it moves onto the guide bars, clips of surgical guide resin are used to avoid slippage as well as to keep the cell fiber in the desired direction as well as reduce on bar displacement.

In some examples, the liquid bath may include calcium chloride. Since the system can use a gravity-based tension control, the dead weight calculation would also account for the gravity with respect to immersion in calcium chloride. The calculated dead weight can be attached between guide bars 2 and 3 and can provide the desired tension in the biological fiber. This weight can be up to 1 gram in some examples. The materials used in this tension control system can be derelin or Delrin and stainless steel. These materials can be used because they can be sterilized. Alongside sterilization, stainless steel also provides good support and easy flow of the fiber on them. These materials can also be non-corrosive which means they can be used for longer exposure in the moisture environment required for this device.

A factor to consider when working with living cells or in this case biological fibers is to keep them hydrated. This can be done because there are specified parameters for each cell to survive with respect to its environment. The cell fibers can be kept wet during the winding process for the cells to survive and/or to promote the crosslinking of the fiber. When the biological fibers used in this application are under a liquid, in this calcium chloride, they are non-adhesive. When they are in contact with air, the biological fibers stick to the required surface very easily. For this reason, the entire tension management system can be completely immersed in liquid.

When the biological fiber is taken from the spool it does not stick to the spool and it can flow freely over the guide bars. Thus, when immersed completely in fluid, the tension management system can provide the desired tension in the biological fiber. In addition to this, the biological fiber sinks in the liquid to enable gravity to aid in providing the desired tension. Once the biological fiber is out of the liquid, it becomes relatively adhesive and hence sticks to the surface of the scaffold or the tissue frame. In this case, there is no need to coat the scaffold or tissue with any extra adhesive coating. However, in other examples, the tissue frame may be coated with another solution or material to assist in friction and adherence of the fiber to the tissue frame.

The bath may have dimensions of 12×8×6 inches is to keep the system compact yet provide enough space to fit the entire tension control system. These dimensions are also chosen to give enough room for the dead weight used to move freely in a vertical motion which at least needs to be 4 inches.

Apart from this the only other liquid parameter used is the part used to stick the first end of the fiber to the scaffold or the organ. For this purpose, a water-based glue can be used. The main reason for using such a glue is to make sure that the starting point of wrapping can be manipulated and can be changed as desired.

The biological fiber that is wound on the surface of the tissue frame may also be kept moist because, if exposed to air for a longer duration of time, the cells within the fiber may dry and die. After a portion of time during the winding, the robotic arm may dip the tissue frame and attached fiber in the liquid bath before proceeding with additional windings. This dipping can provide dynamic hydration of the tissue frame and also aids in the process of quality of the winding. In other examples, a drip or spray system may be used to apply liquid to the fiber on the tissue frame during the winding process.

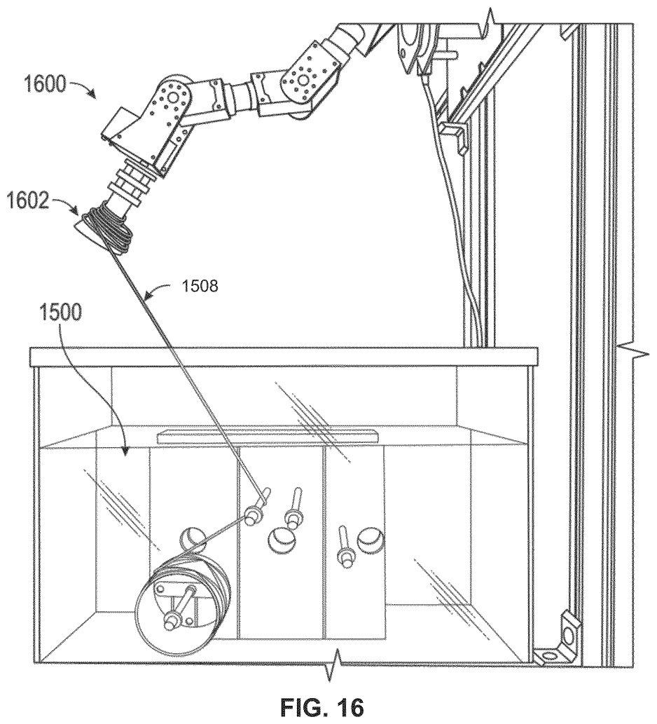

FIG. 16 is a front view of the bath 1500 and example robotic arm 1600 coupled to a tissue frame 1602. In one example, a Hiwonder xArm robot can be controlled by a STM32F103 microcontroller. This microcontroller is based on the ARM Cortex-M3 architecture. This controller can be coded using python. The robot can be programmed to define its trajectory and also can be programmed for various motions and actions. The controller may be programmed to include various positions and trajectories. For example, the controller can define a home position and other positions within the winding process of biological fiber 1508 (beginning, middle, or end), and a trajectory and winding selection algorithm which moves the robotic arm through different angles for one or more of the motors for each respective axis. These trajectories and/or positions may establish the predetermine spatial path that the robot moves the tissue frame through space. In some examples, various positions of each linkage may be programmed to establish the path over time.

From the controls perspective, the freedom to control various parameters related to the winding device can be beneficial. The system may also include a kill switch in case of any failure. For example, the instructions may give the user a choice to select between the types of windings. Each number from 1, 2, or 3 corresponds to LECS, Clasp, and Sling winds respectively. Number 4 corresponds to a dipping action necessary to keep the winding moist. Number 5 corresponds to a combination of all winds and number 6 will terminate any action running in case of any failure. With each selection, the user can also select the number of winds required to be done. The speed for all the actions is set at 550 ms since that is the speed that may be used for biological fiber without any breakage or slippage. This speed selection can be adjusted for other fibers or tissue constructs generated.

As mentioned above, the system is in a liquid held within a glass tank. The polyoxymethylene-based sheet on which the metal bars are integrated is later immersed in this tank and attached to the bottom of the tank with the surgical glue. However, before the immersion of this system into the liquid the materials can be sterilized using autoclaving technique. After the sterilization process, the polyoxymethylene frame is placed into the liquid and the liquid is filled up to a height of 7.5 inches from the bottom of the tank. After this procedure is completed, the cell fiber is attached to the required organ using the glue after which the device is ready for operation.

The system can be controlled by a simple interface. For this device there are two ways in which the user can interact with the device. Either the machine can be controlled with an IDE and then given the inputs for a particular type of winding or it can be accessed via a mobile app in which the inputs can be chosen. As mentioned before, the user can set the angles as well as the rotations based on the type of device. This device is pre coded for winding patterns for the multi axis tool, the GEJ other printed structures. These pre coded patterns can be accessed from the directory available in the app. The parameters can be modified at any given point during the operation.

Time for the winding is also related to the speed at which any action is taking place. For the working of this system, a time is assigned for each action to take place and ultimately for each winding to take place so that a good quality winding can be carried out. The total time taken for one entire winding in all orientations is a combination of various actions as mentioned before. While coding for these windings, a single time delay needs to be given to all the sub actions taking place for a wind. In one example, 50 tests were conducted to optimize the delay time for each action and ultimately the entire time for winding. According to the testing any time delay below 300 milliseconds (ms) per action broke the cell fibers and any time delay above 700 ms per action made the fiber slip or not hold on to the organ frame.

In some examples, a time period of 550 ms per action can be set. With this delay time set, the device can complete the LECS wind in 8 seconds, clasp wind in 11 seconds, and sling wind in 10 seconds thus taking 29 seconds to complete 1 set of winds. If dipping of the organ frame is done, extra 4 seconds are added to this process.

The main aim of this device is to cover the tissue frame as much as possible while still maintaining the alignment of the fibers. The winding device can use a certain length of the fiber to make one single wind around the required axis and make multiple such windings.

After the selection of the trajectory, setting of necessary weights and managing the desired speed, the device can use a minimum of 1.2 inches of cell fiber for the LECS winding and this length can go up to 1.5 inches based on the increasing diameter of the esophagus. For the clasp windings it ranges between 1.7 inches to 2 inches. For the sling winds this length can vary from 1.5 inches to 2.2 inches. Hence for one single set of windings, the average fiber required is 4.4 inches. From these data we can also deduce the total speed of winding is around 0.013 m/s.

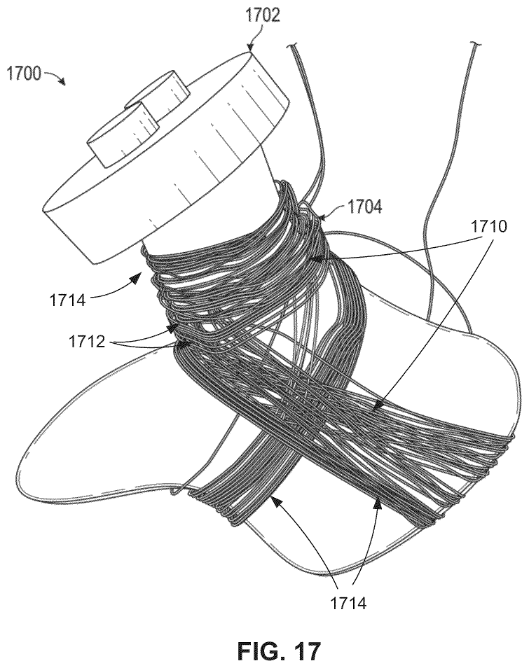

FIG. 17 is an image of an example tissue frame 1700 with string 1704 wound around the tissue frame to illustrate possible directions for a biological fiber on the tissue frame. Attachment device 1702 can be attached to the robotic arm.

In the experiment related to the example of FIG. 17, cotton thread was wound over the tissue frame to test the system. The tension induced with the set trajectory makes the device have a winding that is produced with a certain quality. In the first part of this section, an unmonitored random winding is compared to the winding done with the device and a few results are observed. In the FIG. 17, results of the testing are shown in which blue arrows 1710 represent the hand spun or unsupervised fiber in a single axis. This demonstrates the inaccurate motion as well as random behavior of the winding patterns. The arrows 1712 represent the testing and unoptimized trajectories which were taken into consideration before finalizing the final trajectory, which is shown by arrows 1714. The final trajectory represents the accuracy achieved with the closest optimal trajectory and a weight of 6 grams. These observed windings demonstrate the optimal trajectory of the robot arm after testing 50 times and the increment of the weight after which this accuracy is achieved, thus providing a foundation to the working of this device which is further translated for the actual cell fiber winding.

In another experiment, biological fiber was wound over the organ frame 25 times in each of the three orientations, and it was observed that there is no entanglement and at the same time. The fiber was wound with control and a predicted result is achieved. The trajectory is also repeated perfectly thus providing a proof for repeatability. During this winding experiment, the winding spacing was checked for desired tightness or closeness of the winding. The distance between each winding of the fiber can be selected and determined based on the target tissue construct and/or width of the fiber.

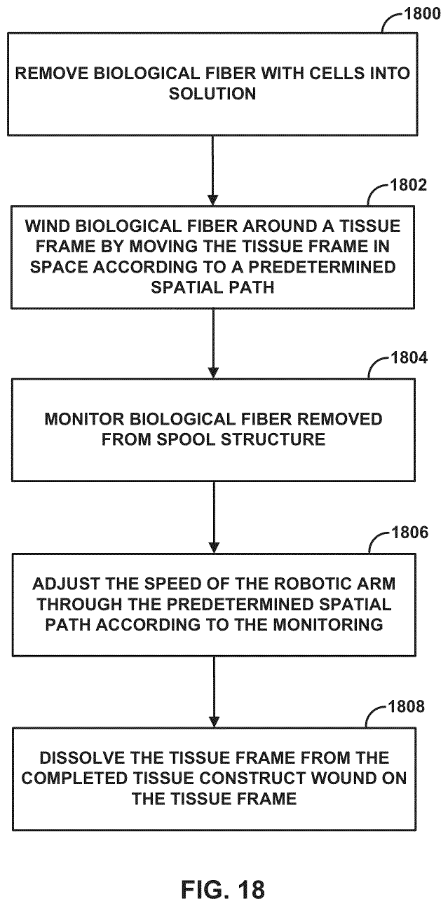

FIG. 18 is a flow diagram of an example technique for winding biological fiber around a tissue frame to form a tissue construct. This process of FIG. 18 may be performed using one or more of systems 600, 630, and 1100. As shown in the example of FIG. 18, the method may include dispensing a biological fiber into a solution, wherein the biological fiber comprises a plurality of cells within a hydrogel (1800). The solution may be a crosslinking bath or other fluid. The dispensing step may be performed by a spinneret or other structure that forms the biological fiber from cells and a gel, for example. The spool structure may be within the solution such that the winding includes pulling the biological fiber off of the spool structure and out of the solution. The removal of the fiber may include a motorized spool to unroll the fiber or a passive spool in which the winding process gradually pulls fiber off of the spool with the tension from winding. In some examples, removing the biological fiber may include rotating, with a motor, the spool structure to expel the biological fiber off of the spool structure and into a solution at a target rate, determining a location of the biological fiber within the solution, and adjusting, based on the location of the biological fiber, at least one a rotational speed of the motor or a winding speed of the robotic arm. In this manner, the system may control how the fiber is removed from the spool structure and maintain a desired condition of the fiber before it is wound on the tissue frame.

Next, the system can use a robotic arm to wind the biological fiber around the tissue frame by moving the tissue frame in space according to a predetermined spatial path (1802). For example, winding the biological fiber may include moving, by the robotic arm having at least six degrees of freedom, the tissue frame in the predetermined path in space to apply the biological fiber to the tissue frame in a complex fiber pattern. This predetermined spatial path may be preprogrammed by the system as the path that the robotic arm may move the tissue frame in 3D space. In some examples, the system may adjust the speed of the movement within the predetermined spatial path. In some examples, the system may make adjustments to the spatial path in order to maintain tension on the biological fiber, adjust for deviations of the fiber being laid into the tissue frame, dip the fiber into a fluid to maintain hydration, or any other reasons.

In the example of FIG. 18, the system monitors the biological fiber removed from the spool structure (1804). For example, the system may monitor the tension of the fiber by monitoring the force acting on the spool structure, a force for a different guide over which the fiber passes, the force of the fiber, and/or an amount of fiber within the solution that has not yet been wound. This monitoring may be to maintain fiber tension onto the tissue frame and/or maintain a desired amount of fiber sitting in the solution (e.g., too little available fiber may cause breakage and too much fiber in solution out of tension may contribute to entanglement of the fiber). The system may monitor the fiber using force sensors, visual sensors, one or more lasers (e.g., laser source and detector to identify laser breaks indicating fiber sinking or migration within the bath), etc. The system can then adjust the speed of the robotic arm through the predetermined spatial path according to the monitoring of the fiber (1806). The example is directed to a tissue frame attached to the robotic arm such that the robotic arm moves the tissue frame with respect to an origin of the biological fiber. In other examples, the tissue frame may be stationary and the robotic arm may move the biological fiber around the tissue frame (either, by moving the fiber in space or moving the spool structure attached to the robotic arm, where the biological fiber is removed from the spool structure as the robotic arm moves). In other examples, the system may move the tissue frame and the biological fiber in space to wind the biological fiber on the tissue frame.

After the system has completed winding of the biological fiber onto the tissue frame, the tissue frame can be dissolved and removed from the tissue construct wound on the tissue frame (1808). For example, the frame may be constructed of a water-soluble material which can be dissolved while maintaining viability of the tissue construct. In other examples, the tissue frame can be peeled away from or separated from the tissue construct, either in pieces or as a single piece.

In some examples, the system may wind the fiber in air, such as above the solution bath from which the fiber is pulled. In other examples, the system may wind the fiber onto the tissue frame within the fluid solution. This can maintain wetness of the fiber while wrapping the fiber. In other examples, the system may partially submerge the tissue frame during the winding process as needed to maintain wetness of the fiber or wick fluid into the frame and/or already wound fiber.

While on the tissue frame, or after the tissue frame is removed (e.g., dissolved, extricated or cut away), the system may mature the biological fiber on the tissue frame. For example, the tissue frame may be placed within a bioreactor so that the cells may stay alive while subjected to the tension applied to the fiber when collected by the tissue frame. In this manner, the tension applied to the biological fiber on the tissue frame can further align the plurality of cells in a direction of the tension.

The system described herein can provide controllability for the parameters of rotations, speed and structure specific winding motion. The system showcases versatility in that it can be used to generate many types of tissue constructs, such as the GEJ and the heart. However, the system can be used to generate any types of tissue constructs, such as portions or tissue or full organs with desired tissue orientations for general biological fabrication.

A user interface that controls the system can have user inputs in which the user can select the type of tissue or scaffold that needs to be wound and the machine should select the necessary code and start the required winding. The UI can also have functionality to control the speed and direction in case a current scaffold or tissue winding procedure needs to be overridden. The system can be fully automated with various levels of intelligence, such as no feedback for adjustment to control, one or more sensors to provide closed-loop control of the winding process, or even anticipated changes based on various feedback during winding. In some examples, the system may include one or more vision sensors from which data can be obtained to control one or more aspects of the robotic device,

From the hardware perspective, the robot arm can use a basic 6 DoF robot. Other types of robots can be used with different levels of precision and accuracy. Instead of using microcontrollers, PLC or industrial state of the art controllers can be used to program the robotic arm for precise windings. For the tension management, instead of just using the guide bars, the system can include an intelligent motor system that can reverse wind the biological fiber to keep the end part of it in tension at all times during the procedure. Such a system can prevent the biological fiber from breaking, and variable tensions can be applied which implies that the strength of the biological fiber can be changed based on the application. A magnet-based system can used for the end effectors, but a biological material may be used to attach the tissue frame or fiber to the robotic arm in other examples.

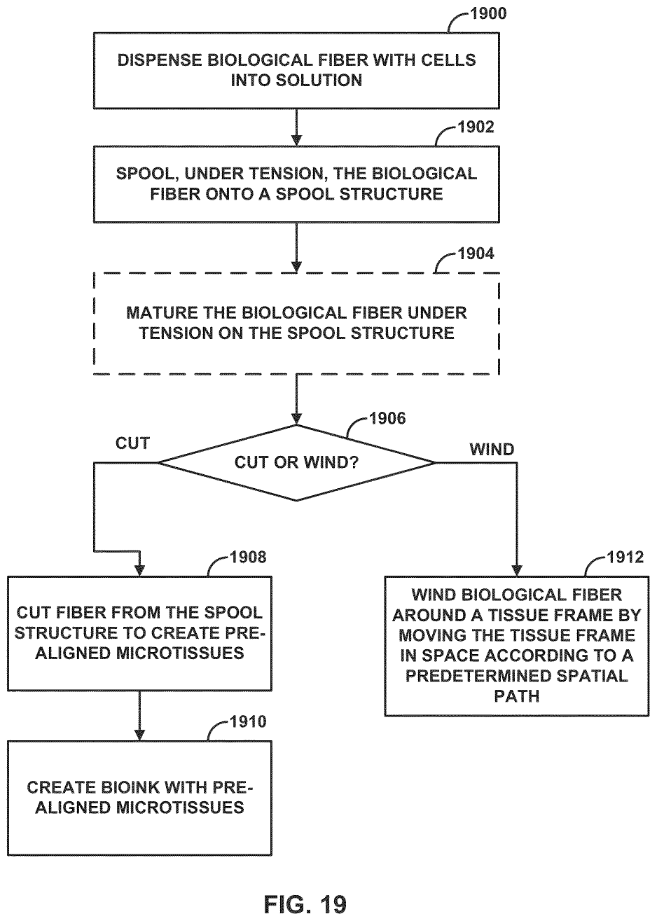

FIG. 19 is a flow diagram of an example technique for creating tissue constructs using spooled biological fiber. The flow diagram of FIG. 19 illustrates how biological fibers may be formed with pre-aligned cells and how the biological fiber can be used for creating tissue constructs. As shown in FIG. 19, a device, such as a spinneret or other device configured to generate the biological fiber that includes cells can dispense the biological fiber with cells in a solution (1900). The system can then spool, under tension, the biological fiber onto a spool structure (1902). This tension may cause the cells to align in the direction of the tension. The system can then mature the biological fiber under the tension on the spool structure such that the cells are fully or partially aligned before the next step (1904). This maturing step may be optional. For example, the maturing process may be performed, or completed, with the biological fiber wound onto the tissue frame.

Once the biological fiber is ready for the next step, the biological fiber can be used for winding into a tissue frame or cut into pre-aligned microtissues for use in a bioink (1906). Of the biological fiber should be cut and used in a bioink, for example, the system can cut the biological fiber that includes aligned cells in the direction of the tension (and along the length of the fiber) to create pre-aligned microtissues (1908). These pre-aligned microstructures can then be added to a liquid, gel, or other substance to create a bioink that includes the pre-aligned microtissues (1910). The bioink can then be printed using a 3D printing nozzle to create 3D tissue constructs. The microtissues can be laid down in the direction of the desired tissue fiber direction which is promoted by the pre-aligned cells within each microtissue in the bioink. Alternatively, the biological fiber can be removed from the spool structure and wound around a tissue frame by moving the tissue frame in space according to a predetermined spatial path (1912). This winding process may be completed by a robotic arm, for example. In some examples, the biological fiber that has been spooled under tension to align the cells in the direction of the tension can be used in other processes that may benefit from the cell alignment created in the biological fiber.

Drug development and disease modeling increasingly rely on small engineered tissues for improved replication of human biology compared to animal models and 2D human cell cultures. While organoids and spheroids (small clumps of cells) in well plates have become somewhat standard, biological fibers are still an emerging technology. Some examples, herein describe large scale manufacturing of repeatable cell fiber fragments is not feasible with current technology. This innovation of generating pre-aligned microtissues addresses both the handling of long fibers and the cutting into repeatable fragments. In some examples, these innovations of generating tissues with complex geometric alignments using winding addresses the handling of long biological fibers.

In some examples, this technology could be used directly in drug development. One example is drug development for cardiac, skeletal, and smooth muscle treatments. All of these tissues are highly organized, which is something that is replicated in biological fibers made of these cells in this method (but not found in current 3D organoids). Increased alignment allows for better modeling of cell communication, especially in the case of cardiac muscle cells being used to model arrhythmias. In other examples, this technology could be used to create tissues for testing medical devices. The examples of spooling technology can also be used in a wide variety of direct patient treatments in the future. Tissue-engineered organs using these cell fibers will not be possible without a system in place to handle the fibers during the production process. The examples of winding technology can also be used in a wide variety of direct patient treatments in the future. Tissue-engineered organs using these biological fibers will not be possible without a system in place to handle the fibers during the production process.

The microtissues and fibers described herein can be made with smooth muscle cells and other cells and biological materials. In some examples, the pre-aligned cell-laden fibers may be used to create or build upon cardiac or skeletal muscle. In some examples, multiple types of microtissues or cellular and acellular fibers can be printed simultaneously, such as to overlap or interdigitate fibers of skeletal muscle and tendon tissue which results in a strong bond between the two tissue types at various tissue type junctions. Creating strong musculotendinous junctions, for example, can be advantageous for treatment of sports injuries, battlefield injuries, and other high energy trauma to soft tissue or other tissues.

The microtissues and fibers described herein may be used in different environments, such as building a larger construct in-vivo or in-vitro. In-vitro construct building may be done prior to implantation of that construct in a subject or otherwise used for a desired purpose. For in-vivo use, the technique may involve directly printing pre-aligned microtissues or depositing fibers to another native tissue. For example, the microtissues or fibers may be deposited onto a native organ to fill a gap in muscle caused by disease or trauma. These new muscle fibers may repair a tear in muscle. This process could also be used for tendons, cardiac muscle, or any other tissue. The system may align the fibers of the printed microtissues with the fibers of the native tissue. In this manner, printing of pre-aligned microtissues or deposition of pre-aligned fibers described herein may be performed to treat injury, repair damage, replace tissue, extend native tissue, or otherwise fix a defect in native subject tissue.

Examples of the disclosure may demonstrate pre-maturing fibers for addressing muscle tissue alignment and vascularization. The examples may provide for rearranging the traditional process flow. In other examples, new methods for creating aligned and vascularized muscle that may be combined and used for further tissue engineering. Some examples of the present disclosure discuss generating, via spinning and spooling long, pre-aligned fibers that can be wound onto a tissue frame. Some examples of the present disclosure discuss generating, via spooling and then cutting biological fibers off of a spool, long, pre-aligned microtissues that can be suspended in a bio-ink for printing using a 3D extrusion bioprinter. These examples may be combined in some examples to achieve the tissues and other structures described herein.

The following examples are described herein.

Example 1. A method comprising: dispensing a biological fiber into a solution, wherein the biological fiber comprises a plurality of cells within a hydrogel; and spooling, under tension, the biological fiber from the solution and onto a spool structure.

Example 2. The method of example 1, wherein the tension applied to the biological fiber on the spool structure at least partially aligns the plurality of cells in a direction of the tension to form a pre-aligned microtissue from the biological fiber.

Example 3. The method of example 2, further comprising: cutting the pre-aligned microtissue from the spool structure into a plurality of pre-aligned microtissues; and suspending the pre-aligned microtissues in a liquid to create a bioink.

Example 4. The method of example 3, further comprising printing a tissue construct by at least depositing the bioink comprising the pre-aligned microtissues in a pattern to create the tissue construct.

Example 5. The method of example 3, wherein the pre-aligned microtissues have a length to width ratio of at least 3:1.

Example 6. The method of any of examples 1 through 5, further comprising increasing at least one dimension of the spool structure that increases the tension applied to the biological fiber on the spool structure.

Example 7. The method of any of examples 1 through 5, further comprising increasing a dimension of an expanding device that contacts the biological fiber on the spool structure to increase the tension applied to the biological fiber.

Example 8. The method of any of examples 1 through 7, further comprising aligning the plurality of cells in a first direction by: applying the tension to the biological fiber; and maturing, with bioreactor signals, the plurality of cells so that the cells are aligned in the first direction to create the pre-aligned microtissue.

Example 9. The method of any of examples 1 through 8, further comprising: measuring the tension applied to the biological fiber during the spooling; and controlling, based on the tension measurement, a speed of the spooling that applies the tension to the biological fiber.

Example 10. The method of any of examples 1 through 9, wherein spooling the biological fiber comprises wrapping the biological fiber around the spool structure within a fluid comprising the biological fiber.

Example 11. The method of any of examples 1 through 10, wherein spooling the biological fiber comprises wrapping the biological fiber around the spool partially submerged in a fluid or completely separate from the fluid, the fluid comprising the biological fiber.

Example 12. The method of any of examples 1 through 11, further comprising: embedding the spool structure with the biological fiber into a gel; cutting the gel and embedded spooled biological fiber into a plurality of pre-aligned microtissues; and removing the plurality of pre-aligned microtissues from the gel by melting the gel from the plurality of pre-aligned microtissues.

Example 13. The method of any of examples 1 through 12, further comprising: unspooling, from the spool structure, the biological fiber to a drawing roller; and cutting the biological fiber on the drawing roller into a plurality of pre-aligned microtissues.

Example 14. The method of any of examples 1 through 13, further comprising: removing the biological fiber from the spool structure; and winding, with a robotic arm, the biological fiber around a tissue frame to form a tissue construct.

Example 15. The method of example 14, further comprising: measuring tension applied to the biological fiber during the winding; and controlling, based on the tension measurement, a speed of movement of the robotic arm that applies the tension to the biological fiber.

Example 16. The method of any of examples 14 and 15, wherein the spool structure is disposed within a solution, and wherein the winding comprises pulling the biological fiber off of the spool structure and out of the solution.

Example 17. The method of any of examples 14 through 16, wherein removing the biological fiber comprises: rotating, with a motor, the spool structure to expel the biological fiber off of the spool structure and into a solution at a target rate; determining a location of the biological fiber within the solution; and adjusting, based on the location of the biological fiber, at least one a rotational speed of the motor or a winding speed of the robotic arm.

Example 18. The method of any of examples 14 through 17, further comprising applying a fluid to the biological fiber around the tissue frame during the winding.

Example 19. The method of any of examples 14 through 18, wherein winding the biological fiber comprises moving, by the robotic arm having at least six degrees of freedom, the tissue frame in a predetermined path in space to apply the biological fiber to the tissue frame in a complex fiber pattern.

Example 20. The method of any of examples 14 through 19, further comprising maturing, with bioreactor signals, the plurality of pre-aligned cells of the biological fiber wound around the tissue frame so that the cells further align in a direction of tension applied during the winding to create the complex aligned tissue.

Example 21. The method of any of examples 14 through 20, wherein the biological fiber wound around the tissue frame comprises the tissue construct in the form of a target organ.

Example 22. The method of any of examples 14 through 21, further comprising dissolving the tissue frame from the wound biological fiber to remove the tissue construct from the tissue frame.

Example 23. The method of any of examples 14 through 22, further comprising expanding the tissue frame in at least one dimension to increase tension on at least a portion of the biological fiber wound around the tissue frame.

Example 24. The method of any of examples 14 through 23, wherein winding comprises winding, with the robotic arm attached to the tissue frame, the biological fiber around the tissue frame by moving the tissue frame with respect to the spool structure.

Example 25. The method of any of examples 1 through 24, wherein the one or more cells are gut smooth muscle cells (gSMC).

Example 26. A system configured to perform the method of any of examples 1 through 25.

Example 27. A system comprising: a spinneret configured to dispense a biological fiber into a solution, wherein the biological fiber comprises a plurality of cells within a hydrogel; a spool structure configured to store the biological structure; and a motor coupled to at least one of a roller or the spool structure and configured to apply tension to the biological fiber, wherein the motor is configured to assist in spooling, under the tension, the biological fiber from the solution and onto the spool structure.

Example 28. The system of example 27, further comprising a controller configured to control the motor to apply the tension to the biological fiber on the spool structure such that the tension at least partially aligns the plurality of cells in a direction of the tension to form a pre-aligned microtissue from the biological fiber.

Example 29. The system of any of examples 27 or 28, further comprising a cutting tool configured to cut the pre-aligned microtissue from the spool structure into a plurality of pre-aligned microtissues, wherein the pre-aligned microtissues are configured to be suspended in a liquid to create a bioink.

Example 30. The system of example 29, further comprising a printing nozzle configured to print a tissue construct by at least depositing the bioink comprising the pre-aligned microtissues in a pattern to create the tissue construct.

Example 31. The system of example 29, wherein the pre-aligned microtissues have a length to width ratio of at least 3:1.

Example 32. The system of any of examples 27 through 31, wherein the spool comprises an expandable device configured to increasing at least one dimension of the spool structure that increases the tension applied to the biological fiber on the spool structure.

Example 33. The system of any of examples 27 through 31, further comprising an expandable device configured to increase a dimension of the expanding device that contacts the biological fiber on the spool structure to increase the tension applied to the biological fiber.

Example 34. The system of any of examples 27 through 33, wherein at least the roller or the spool structure is configured to align the plurality of cells in a first direction by at least: applying the tension to the biological fiber; and maturing, with bioreactor signals, the plurality of cells so that the cells are aligned in the first direction to create the pre-aligned microtissue.

Example 35. The system of any of examples 27 through 34, further comprising: a load cell configured to measure the tension applied to the biological fiber during the spooling; and a controller configured to control, based on the tension measurement, a speed of the spooling that applies the tension to the biological fiber.

Example 36. The system of any of examples 27 through 35, wherein the spool structure is configured to wrap the biological fiber around the spool structure within a fluid comprising the biological fiber.

Example 37. The system of any of examples 27 through 36, wherein the spool structure is configured to wrap the biological fiber around the spool partially submerged in a fluid or completely separate from the fluid, the fluid comprising the biological fiber.

Example 38. The system of any of examples 27 through 36, further comprising: a drawing roller configured to unspool, from the spool structure, the biological fiber to the drawing roller; and a cutting device configured to cut the biological fiber on the drawing roller into a plurality of pre-aligned microtissues.

Example 39. The system of any of examples 27 through 38, further comprising a robotic arm configured to wind, from the spool structure, the biological fiber around a tissue frame to form a tissue construct.

Example 40. The system of example 39, further comprising: a load cell configured to measure tension applied to the biological fiber during the winding; and control, based on the tension measurement, a speed of movement of the robotic arm that applies the tension to the biological fiber.

Example 41. The system of any of examples 39 and 40, wherein the spool structure is disposed within a solution, and wherein the robotic arm is configured to remove the biological fiber from the spool structure by at least pulling the biological fiber off of the spool structure and out of the solution.

Example 42. The system of any of examples 39 through 41, further comprising: a motor configured to rotate the spool structure to expel the biological fiber off of the spool structure and into a solution at a target rate; and a controller configured to: determine, from at least one sensor, a location of the biological fiber within the solution; and adjust, based on the location of the biological fiber, at least one a rotational speed of the motor or a winding speed of the robotic arm.

Example 43. The system of any of examples 39 through 42, further comprising a nozzle configured to apply a fluid to the biological fiber around the tissue frame during the winding.

Example 44. The system of any of examples 39 through 43, wherein the robotic arm is configured to wind the biological fiber by at least moving, by the robotic arm having at least six degrees of freedom, the tissue frame in a predetermined path in space to apply the biological fiber to the tissue frame in a complex fiber pattern.

Example 45. The system of any of examples 39 through 44, further comprising a bioreactor configured to mature, with bioreactor signals, the plurality of pre-aligned cells of the biological fiber wound around the tissue frame so that the cells further align in a direction of tension applied during the winding to create the complex aligned tissue.

Example 46. The system of any of examples 39 through 45, wherein the biological fiber wound around the tissue frame comprises the tissue construct in the form of a target organ.

Example 47. The system of any of examples 39 through 46, wherein the tissue frame is configured to expand in at least one dimension to increase tension on at least a portion of the biological fiber wound around the tissue frame.

Example 48. The system of any of examples 39 through 47, wherein the robotic arm is configured to wind, with the robotic arm attached to the tissue frame, the biological fiber around the tissue frame by at least moving the tissue frame with respect to the spool structure.

Example 49. The system of any of examples 27 through 48, wherein the one or more cells are gut smooth muscle cells (gSMC).

Example 101. A method comprising: dispensing a biological fiber into a solution, wherein the biological fiber comprises a plurality of cells within a hydrogel; and spooling, under tension, the biological fiber from the solution and onto a spool structure.

Example 102. The method of example 101, wherein the tension applied to the biological fiber on the spool structure at least partially aligns the plurality of cells in a direction of the tension to form a pre-aligned microtissue from the biological fiber.

Example 103. The method of example 102, further comprising: cutting the pre-aligned microtissue from the spool structure into a plurality of pre-aligned microtissues; printing a tissue construct by at least: suspending the pre-aligned microtissues in a liquid to create a bioink; and depositing the pre-aligned microtissues in a second direction to create the tissue construct.

Example 104. The method of example 103, wherein the pre-aligned microtissues have a length to width ratio of at least 3:1.

Example 105. The method of any of examples 101 through 104, further comprising aligning the plurality of cells in a first direction by: applying the tension to the biological fiber; and maturing, with bioreactor signals, the plurality of cells so that the cells are aligned in the first direction to create the pre-aligned microtissue.

Example 106. The method of any of examples 101 through 105, further comprising: measuring the tension applied to the biological fiber during the spooling; and controlling. based on the tension measurement, a speed of the spooling that applies the tension to the biological fiber.

Example 107. The method of any of examples 101 through 106, wherein spooling the biological fiber comprises wrapping the biological fiber around the spool structure within a fluid comprising the biological fiber.

Example 108. The method of any of examples 101 through 107, wherein spooling the biological fiber comprises wrapping the biological fiber around the spool partially submerged in a fluid or completely separate from the fluid, the fluid comprising the biological fiber.

Example 109. The method of any of examples 101 through 108, further comprising: embedding the spool structure with the biological fiber into a gel; cutting the gel and embedded spooled biological fiber into a plurality of pre-aligned microtissues; and removing the plurality of pre-aligned microtissues from the gel by melting the gel from the plurality of pre-aligned microtissues.

Example 110. The method of any of examples 101 through 109, further comprising: unspooling, from the spool structure, the biological fiber to a drawing roller; and cutting the biological fiber on the drawing roller into a plurality of pre-aligned microtissues.

111. The method of any of examples 101 through 110, wherein the one or more cells are gut smooth muscle cells (gSMC).

Example 112. A system configured to perform the method of any of examples 101 through 111.

Example 113. A system comprising: a spinneret configured to dispense a biological fiber into a solution, wherein the biological fiber comprises a plurality of cells within a hydrogel; a spool structure configured to store the biological structure; and a motor coupled to at least one of a roller or the spool structure and configured to apply tension to the biological fiber, wherein the motor is configured to assist in spooling, under the tension, the biological fiber from the solution and onto the spool structure.

Example 201. A method comprising: removing a biological fiber from a spool structure, wherein the biological fiber comprises a plurality of cells within a hydrogel; and winding, with a robotic arm, the biological fiber around a tissue frame.

Example 202. The method of example 201, further comprising: measuring tension applied to the biological fiber during the winding; and controlling, based on the tension measurement, a speed of movement of the robotic arm that applies the tension to the biological fiber.

Example 203. The method of any of examples 201 and 202, wherein the spool structure is disposed within a solution, and wherein the winding comprises pulling the biological fiber off of the spool structure and out of the solution.

Example 204. The method of any of examples 201 through 203, wherein removing the biological fiber comprises: rotating, with a motor, the spool structure to expel the biological fiber off of the spool structure and into a solution at a target rate; determining a location of the biological fiber within the solution; and adjusting, based on the location of the biological fiber, at least one a rotational speed of the motor or a winding speed of the robotic arm.

Example 205. The method of any of examples 201 through 204, further comprising applying a fluid to the biological fiber around the tissue frame during the winding.

Example 206. The method of any of examples 201 through 205, wherein winding the biological fiber comprises moving, by the robotic arm having at least six degrees of freedom, the tissue frame in a predetermined path in space to apply the biological fiber to the tissue frame in a complex fiber pattern.

Example 207. The method of any of examples 201 through 206, further comprising maturing, with bioreactor signals, the plurality of pre-aligned cells of the biological fiber wound around the tissue frame so that the cells further align in a direction of tension applied during the winding to create the complex aligned tissue.

Example 208. The method of any of examples 201 through 207, wherein the biological fiber wound around the tissue frame comprises a tissue construct in the form of a target organ.

Example 209. The method of any of examples 201 through 208, further comprising dissolving the tissue frame from the wound biological fiber to create a tissue construct.

Example 210. The method of any of examples 201 through 209, wherein tension applied to the biological fiber on the spool structure at further aligns the plurality of cells in a direction of the tension to form a complex aligned tissue from the biological fiber.

Example 211. The method of any of examples 201 through 210, wherein winding comprises winding, with the robotic arm attached to the tissue frame, the biological fiber around the tissue frame by moving the tissue frame with respect to the spool structure.

Example 212. The method of any of examples 201 through 211, wherein the one or more cells could be gut smooth muscle cells (gSMC).

Example 13. A system configured to perform the method of any of examples 201 through 212.

Example 214. A system comprising: memory configured to store a predetermined spatial path; and control circuitry configured to: control a spool structure to remove a biological fiber from the spool structure, wherein the biological fiber comprises a plurality of cells within a hydrogel; and control a robotic arm to wind the biological fiber around the tissue frame by moving the biological fiber with respect to the tissue frame in the predetermined spatial path.

Example 215. A system comprising: memory configured to store a predetermined spatial path; and control circuitry configured to: control a spool structure to remove a biological fiber from the spool structure, wherein the biological fiber comprises a plurality of cells within a hydrogel; and control a robotic arm attached to a tissue frame to wind the biological fiber around the tissue frame by moving the tissue frame with respect to the spool structure in the predetermined spatial path.

The techniques described in this disclosure may be implemented, at least in part, in hardware, software, firmware or any combination thereof. For example, various aspects of the described techniques may be implemented within one or more processors, such as fixed function processing circuitry and/or programmable processing circuitry, including one or more microprocessors, digital signal processors (DSPs), application specific integrated circuits (ASICs), field programmable gate arrays (FPGAs), or any other equivalent integrated or discrete logic circuitry, as well as any combinations of such components. The term “processor” or “processing circuitry” may generally refer to any of the foregoing logic circuitry, alone or in combination with other logic circuitry, or any other equivalent circuitry. A control unit comprising hardware may also perform one or more of the techniques of this disclosure.

Such hardware, software, and firmware may be implemented within the same device or within separate devices to support the various operations and functions described in this disclosure. In addition, any of the described units, modules or components may be implemented together or separately as discrete but interoperable logic devices. Depiction of different features as modules or units is intended to highlight different functional aspects and does not necessarily imply that such modules or units must be realized by separate hardware or software components. Rather, functionality associated with one or more modules or units may be performed by separate hardware or software components, or integrated within common or separate hardware or software components.

The techniques described in this disclosure may also be embodied or encoded in a computer-readable medium, such as a computer-readable storage medium, containing instructions. Instructions embedded or encoded in a computer-readable storage medium may cause a programmable processor, or other processor, to perform the method, e.g., when the instructions are executed. Computer readable storage media may include random access memory (RAM), read only memory (ROM), programmable read only memory (PROM), erasable programmable read only memory (EPROM), electronically erasable programmable read only memory (EEPROM), flash memory, a hard disk, a CD-ROM, a floppy disk, a cassette, magnetic media, optical media, or other computer readable media.

Various aspects of the disclosure have been described. These and other aspects are within the scope of the following claims.

1. A method comprising:
dispensing a biological fiber into a solution, wherein the biological fiber comprises a plurality of cells within a hydrogel; and
winding under tension, the biological fiber from the solution and onto a structure.

2. The method of claim 1, wherein the tension applied to the biological fiber on the spool structure at least partially aligns the plurality of cells in a direction of the tension to form a pre-aligned tissue from the biological fiber.

3. The method of claim 1, wherein:
the structure comprises a spool structure,
winding the biological fiber comprises spooling the biological fiber onto the spool structure, and
the method further comprises:
cutting the biological fiber from the spool structure into a plurality of microtissues; and
suspending the microtissues in a liquid to create a bioink.

4-6. (canceled)

7. The method of claim 1, further comprising increasing a dimension of one of an expanding device that contacts the biological fiber on the structure or the structure itself to increase the tension applied to the biological fiber.

8. The method of claim 1, further comprising aligning the plurality of cells in a first direction by:
applying the tension to the biological fiber; and
maturing, with bioreactor signals, the plurality of cells so that the cells are aligned in the first direction to create a pre-aligned tissue.

9. The method of claim 1, further comprising:
measuring the tension applied to the biological fiber during the winding; and
controlling, based on the tension measurement, a speed of the winding that applies the tension to the biological fiber.

10. The method of claim 1, wherein winding the biological fiber comprises wrapping the biological fiber around the structure within a fluid comprising the biological fiber.

11-13. (canceled)

14. The method of claim 1, wherein the structure comprises a spool structure, and wherein the method further comprises:
removing the biological fiber from the spool structure; and
winding, with a robotic arm, the biological fiber around a tissue frame to form a tissue construct.

15. The method of claim 1, wherein the structure comprises a tissue frame, and wherein the method further comprises:
measuring tension applied to the biological fiber during the winding; and
controlling, based on the tension measurement, a speed of movement of a robotic arm that applies the tension to the biological fiber during winding of the biological fiber around the tissue frame.

16. (canceled)

17. The method of claim 14, wherein removing the biological fiber comprises:
rotating, with a motor, the spool structure to expel the biological fiber off of the spool structure and into a solution at a target rate;
determining a location of the biological fiber within the solution; and
adjusting, based on the location of the biological fiber, at least one a rotational speed of the motor or a winding speed of the robotic arm.

18. The method of claim 1, wherein the structure comprises a tissue frame, wherein the winding comprises winding, with a robotic arm, the biological fiber around the tissue frame to form a tissue construct, and wherein the method further comprises applying a fluid to the biological fiber around the tissue frame during the winding.

19. The method of claim 1, wherein the structure comprises a tissue frame, wherein winding the biological fiber onto the tissue frame comprises moving, by a the robotic arm having at least six degrees of freedom, the tissue frame in a predetermined path in space to apply the biological fiber to the tissue frame in a complex fiber pattern.

20. The method of claim 1, wherein the structure comprises a tissue frame, wherein the method further comprises maturing, with bioreactor signals, the plurality of cells of the biological fiber wound around the tissue frame so that the cells align in a direction of tension applied during the winding to create the complex aligned tissue, and wherein the complex aligned tissue is in a form of a target organ.

21. (canceled)

22. The method of claim 18, further comprising dissolving the tissue frame from the wound biological fiber to remove the tissue construct from the tissue frame.

23-24. (canceled)

25. The method of claim 1, wherein the plurality of cells comprise one or more of gut smooth muscle cells (gSMC), skeletal muscle cells, or cardiac smooth muscle cells.

26. (canceled)
